SEQUENCE LISTING (17 sequences)

SEQ ID NO: 1 - DNA, 36 bp, Artificial Sequence
  Primer
ccgtcgttttacaacgtcgtgactgggaaaaccctg

SEQ ID NO: 2 - DNA, 34 bp, Artificial Sequence
  Primer
tatacgactcactataggccattcgccattcagg

SEQ ID NO: 3 - DNA, 20 bp, Artificial Sequence
  Primer
cccctgtgaggaactactgt

SEQ ID NO: 4 - DNA, 20 bp, Artificial Sequence
  Primer
ggtgcacggtctacgagacc

SEQ ID NO: 5 - DNA, 32 bp, Artificial Sequence
  Primer
gggtcgcgaaaggccttgtggtactgcctgat

SEQ ID NO: 6 - DNA, 30 bp, Artificial Sequence
  Primer
gttgcatagttcacgccgtcttccagaacc

SEQ ID NO: 7 - DNA, 40 bp, Artificial Sequence
  Primer
gcgtccgggttctggaagacggcgtgaactatgcaacagg

SEQ ID NO: 8 - DNA, 40 bp, Artificial Sequence
  Primer
aggctttcattgcagttcaaggccgtgctattgatgtgcc

SEQ ID NO: 9 - DNA, 20 bp, Artificial Sequence
  Primer
tgacgtccatgctcactgat

SEQ ID NO: 10 - DNA, 20 bp, Artificial Sequence
  Primer
gagacttccgcaggatttct

SEQ ID NO: 11 - DNA, 31 bp, Artificial Sequence
  Primer
tggggatcccgtatgatacccgctgctttga

SEQ ID NO: 12 - DNA, 30 bp, Artificial Sequence
  Primer
ggcggaattcctggtcatagcctccgtgaa

SEQ ID NO: 13 - DNA, 263 bp, Hepatitis C virus
  Synthetic construct
cttcacgcagaaagcgtctagccatggagttagtatgagtgtcgtgcagcctccaggaccccccctcccgggaggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccggga

SEQ ID NO: 14 - protein, 151 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARAL

SEQ ID NO: 15 - protein, 160 aa, Hepatitis C virus
SALYVGDLCGSVFLVGQLFTFSPRRHWTTQDCNCSIYPGHITGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALN

SEQ ID NO: 16 - protein, 79 aa, Hepatitis C virus
TSMLTDPSHITAEAAGRRLARGSPPSMASSSASQLSAPSLKATCTANHDSPDAELIEANLLWRQEMGGNITRVESENKV

SEQ ID NO: 17 - protein, 120 aa, Hepatitis C virus
RCFDSTVTESDIRTEEAIYQCCDMDPQARVAIKSLTERLYVGGPLTNSRGENCGYRRCRASGVLTTSCGNTLTCYIKARAAGLQDCTMLVCGDDLVVICESAGVQEDAASLRAFTEAMTR

(19) United States
(12) Patent Application Publication
(10) Pub. No.: US 2003/0148267 A1
(43) Pub. Date: Aug. 7, 2003

(54) Screening assay for hepatitis C virus antiviral agents

(76) Inventors: Emmett Vance Schmidt, Andover, MA (US); Raymond Taeyong Chung, Chestnut Hill, MA (US)

Correspondence Address: JANIS K. FRASER, PH.D., J.D., Fish & Richardson P.C., 225 Franklin Street, Boston, MA, 02110-2804, US

(21) Appl. No.: 10/292,129

(22) Filed: Nov. 8, 2002

Related U.S. Application Data
(60) Provisional application No. 60/345,405, filed on Nov. 9, 2001.

(51) Int. Cl. C12Q 1/70; C12Q 1/68; C12P 19/34

(52) U.S. Cl. 435/5; 435/6; 435/91.2

(57) ABSTRACT

The invention includes methods of identifying compounds that increase the mutation rate of hepatitis C virus. The invention can be used to screen libraries of test compounds, including both nucleoside analogs and non-nucleoside analogs. The methods include: 1) contacting a test cell with a candidate compound, wherein the test cell contains a nucleic acid molecule comprising an infectious hepatitis C viral genome, a ribozyme, and an inducible promoter operably linked to the first and second nucleotide sequences, the ribozyme being configured to remove a 3′ sequence unnecessary for replication of the infectious hepatitis C viral genome from a transcript initiated by the inducible promote; and 2) the detection of an increase in hepatitis C virus quasispecies produced by the cell in the presence of the candidate compound. Detection of an increase in quasispecies can be accomplished by, e.g., sequencing HCV nucleic acid molecules isolated from the test cell.

### RELATED APPLICATION INFORMATION

This application claims priority from provisional application serial No. 60/345,405, filed Nov. 9, 2001, the contents of which are hereby incorporated by reference.

### STATEMENT AS TO FEDERALLY SPONSORED RESEARCH

The invention was made with Government support under NIH Grant Nos. R01 DK 57857-02 and AI XXXX. The Government therefore has certain rights in this invention.

### TECHNICAL FIELD

This invention relates to screening assays for antiviral therapeutics.

### BACKGROUND

Infection with hepatitis C virus (HCV) is one of the leading causes of chronic liver disease throughout the world. Chronic infection nearly always ensues after acute exposure to HCV, and chronically infected individuals develop cirrhosis and hepatocellular carcinoma at a dramatically elevated rate compared with the rate of those diseases in an uninfected population.

HCV is a positive strand RNA virus. Its genome encodes a single precursor viral protein that is cleaved by cellular and viral proteases to generate viral structural and non-structural proteins, respectively. Short non-translated regions located 5′ and 3′ (5′NTR and 3′NTR) to the precursor protein open reading frame are involved in translation initiation and viral replication, respectively. For a review of HCV, see Houghton, “Chapter 32, Hepatitis C Viruses,” in: Fields Virology, 3rd ed., Fields et al. eds., pp 1035-1058, 1996, Lippincott-Raven Publishers, Philadelphia, Pa.

HCV has a high mutation rate, which helps the virus elude its host's immune system. The high mutation rate is reflected in the presence of many distinct HCV genome sequences, known as quasispecies, within infected individuals. Quasispecies result from the activity of the virally-encoded NS5B RNA-dependent RNA polymerase, which, because of its inherent lack of proofreading function, is a low-fidelity enzyme.

Sequence heterogeneity has been described throughout the coding regions of the HCV genome. In contrast, the 5′ and 3′ untranslated regions display exceptional sequence conservation, which is probably a consequence of the critical role these regions play in translation initiation and RNA replication. The most variable regions of the genome are situated in regions associated with B-cell epitopes, strongly suggesting that immune selection pressure plays a significant role in quasispecies diversity. However, until recently, the lack of a tractable in vitro HCV replication system has made it difficult to distinguish between error generation attributable to the NS5B polymerase and errors resulting from immune selection pressure.

For RNA viruses that exhibit a high mutation rate, it has been proposed that a moderate increase in the mutation rate may kill a virus population by causing an “error catastrophe”. In this regard, ribavirin (RBV), a guanosine analogue, is an approved agent for the treatment of hepatitis C virus, when used in conjunction with interferon-α. RBV has been proposed to act as a direct antiviral agent by acting to deplete guanosine nucleoside pools and inhibit viral transcription. Furthermore, for poliovirus, RBV is believed to act as an RNA mutagen (see Crotty et al. (2000), Nature Medicine 6(12):1375-9). The mechanism of RBV's antiviral effect against HCV, though, remains unknown.

Methods for determining the mutation rate of HCV in the absence of selective pressures generated by host immune response would be highly beneficial, as they could be used to identify compounds that increase the mutation rate. Such compounds may induce an error catastrophe and thus have value as potential therapeutic agents useful for the treatment of HCV infection.

### SUMMARY

To address the need for a method of determining the mutation rate of hepatitis C virus (HCV), the invention provides an inducible in vitro HCV replication system and methods for analyzing and quantifying the production of HCV quasispecies (i.e., HCV quasispecie nucleic acids and viral particles) in the replication system. Thus, the invention is based, in part, on the development of an in vitro cell-based system for inducing HCV replication and producing hepatitis C nucleic acids and viral particles, including HCV quasispecie nucleic acids and viral particles. The invention is also based, in part, on methods of using the in vitro HCV replication system to assay for and quantify the production of HCV quasispecies.

Accordingly, in one aspect the invention features nucleic acid molecules comprising a first nucleotide sequence encoding an infectious hepatitis C viral genome, a second nucleotide sequence encoding a ribozyme (e.g., a hepatitis D virus ribozyme), an inducible promoter (e.g., a T7 promoter) operably linked to the first and second nucleotide sequences, and optionally a transcription termination signal (e.g., a T7 transcription termination signal) operably linked to the first and second nucleotide sequences, the ribozyme being configured to remove a 3′ sequence unnecessary for replication of the infectious hepatitis C virus from a transcript initiated by the inducible promoter and optionally terminated by the transcription termination signal. The invention also includes cells that harbor a nucleic acid molecule of the invention.

In another aspect, the invention features a method of producing HCV nucleic acid molecules, e.g., plus and minus strand HCV RNA, and HCV viral particles, including HCV quasispecie nucleic acid molecules and viral particles. In preferred embodiments, a cell containing a nucleic acid molecule of the invention (e.g., as an episome or an integrated cassette) is used to generate HCV quasispecie nucleic acids and viral particles by inducing the inducible promoter of the nucleic acid molecule. For example, if the promoter is a T7 bacteriophage promoter, HCV quasispecie nucleic acids and viral particles are produced by expressing a T7 RNA polymerase in the cell. In some embodiments, the T7 RNA polymerase is expressed by infecting the cell with a viral vector (e.g., a vaccinia vector) encoding the T7 RNA polymerase. In other embodiments, the cell contains an episomal plasmid or genomic transgene (e.g., delivered by a retrovirus) that expresses T7 RNA polymerase. Regardless of the vectors used to express T7 RNA polymerase, the expression of the polymerase can itself be regulated, depending on the genetic elements operably linked to the sequence encoding the polymerase.

As used herein, an “induced cell” is a cell that contains a nucleic acid molecule of the invention in which HCV replication has been induced (i.e., the cell is producing plus and minus strand HCV RNA molecules).

In some embodiments, HCV quasispecie nucleic acids and viral particles are produced by passaging infectious HCV viral particles produced by an induced cell. In preferred embodiments, the passaging process comprises contacting cells that lack replicative HCV nucleic acid molecules (i.e., plus or minus strand HCV RNA) with the infectious HCV viral particles and allowing the cells to be infected by the HCV viral particles, whereby HCV replication occurs and HCV quasispecie nucleic acids and viral particles are produced by the cells. In some embodiments, the cells being contacted contain a nucleic acid molecule of the invention. In other embodiments, the cells being contacted do not contain a nucleic acid molecule of the invention. In some embodiments, obtaining infectious HCV viral particles comprises obtaining cell culture medium used to culture at least one induced cell. In preferred embodiments, the cell culture medium is substantially free of cells (e.g., the cell culture medium has been centrifuged and/or filtered to remove cells). Such cell culture medium can be used to contact cells that lack replicative HCV nucleic acid molecules and are permissive to HCV infection. In other embodiments, obtaining infectious HCV viral particles comprises obtaining at least one induced cell. The induced cell can be mixed with the cells being contacted, wherein the mixing provides the means of contacting the cells with an infectious HCV viral particle. In preferred embodiments, the induced cell is mixed with the cells being contacted after inducing HCV replication in the induced cell. In other embodiments, the induced cell is mixed with the cells being contacted before inducing HCV replication in the induced cell.

In another aspect, the invention features a method of identifying and/or quantifying HCV quasispecies, i.e., HCV plus or minus strand nucleic acid molecules and/or HCV viral particles, produced in the in vitro HCV replication system, e.g., HCV quasispecie nucleic acids and viral particles produced in an induced cell or in a cell that has been infected by an HCV viral particle produced by an induced cell. In some embodiments, the HCV quasispecie nucleic acid molecule is identified in a collection of HCV nucleic acid molecules, e.g., plus or minus strand HCV RNA molecules, obtained from a cell, e.g., an induced cell. In other embodiments, the HCV quasispecie nucleic acid molecule is identified in a collection of HCV nucleic acid molecules, e.g., plus or minus strand HCV RNA molecules, obtained from viral particles, e.g., HCV viral particles produced by an induced cell.

In some embodiments, the method comprises amplifying, e.g., by RT-PCR, HCV nucleic acid molecules, e.g., plus or minus strand HCV RNA molecules or any portion thereof, present in a cell, e.g., an induced cell, or viral particle, e.g., a viral particle produced by an induced cell. In preferred embodiments, the method comprises amplifying, e.g., by RT-PCR, a fragment of a HCV plus or minus strand RNA molecule, e.g., a fragment comprising an HCV non-coding region (e.g., a 5′UTR or 3′UTR region) or an HCV protein coding region, e.g., a core, E1, E2, NS2, NS3, NS4a, NS4b, NS5a, or NS5b coding region (or fragment thereof). In particularly preferred embodiments, the method comprises amplifying a fragment comprising a 5′UTR region or a core, E1, E2, NS5a, or NS5b coding region.

In some embodiments, the method comprises determining the sequence of an HCV nucleic acid molecule, e.g., an HCV plus or minus strand RNA molecule or fragment thereof, present in a cell, e.g., an induced cell, or viral particle, e.g., a viral particle produced by an induced cell. In preferred embodiments, the HCV nucleic acid molecule is a fragment, e.g., a fragment comprising an HCV non-coding region (e.g., a 5′UTR or 3′UTR region) or an HCV protein coding region, e.g., a core, E1, E2, NS2, NS3, NS4a, NS4b, NS5a, or NS5b coding region, or portion thereof. In some embodiments, the HCV nucleic acid molecule has been amplified, e.g., by RT-PCR. In preferred embodiments, the sequence of more than one HCV nucleic acid molecule is determined. In particularly preferred embodiments, the sequences of at least 5, 10, 15, 20, or more individual HCV nucleic acid molecules are determined. In some embodiments, the sequences of two or more individual HCV nucleic acid molecules are determined collectively, e.g., a single sequencing reaction can be performed on a set of HCV nucleic acid molecules wherein the set contains a plurality of unique HCV nucleic acid sequences. While this approach cannot provide sequences for each individual molecule, it can indicate positions in the sequence that vary between molecules. In preferred embodiments, the sequences of two or more HCV nucleic acid molecules are determined individually, e.g., the HCV nucleic acid molecules can be cloned and the sequence of each individual clone can be determined.

In other embodiments, the method comprises analyzing an HCV nucleic acid molecule, e.g., a plus or minus strand RNA molecule or fragment thereof, obtained from a cell, e.g., an induced cell, or viral particle, e.g., a viral particle produced by an induced cell, using a heteroduplex mobility assay. In preferred embodiments, the HCV nucleic acid molecule is a fragment, e.g., a fragment comprising an HCV non-coding region (e.g., a 5′UTR or 3′UTR region) or an HCV protein coding region, e.g., a core, E1, E2, NS2, NS3, NS4a, NS4b, NS5a, or NS5b coding region. In some embodiments, the HCV nucleic acid molecule has been amplified, e.g., by RT-PCR. In preferred embodiments, the heteroduplex mobility assay is performed on more than one HCV nucleic acid molecule. In particularly preferred embodiments, the heteroduplex mobility assay is performed on at least 5, 10, 15, 20, or more individual HCV nucleic acid molecules. In some embodiments, RFLP analysis is performed on a plurality of HCV nucleic acid molecules simultaneously, e.g., a single heteroduplex mobility assay can be performed on a set of HCV nucleic acid molecules wherein the set contains multiple unique HCV nucleic acid sequences. In preferred embodiments, the heteroduplex mobility assay is performed on two or more HCV nucleic acid molecules individually, e.g., the HCV nucleic acid molecules can be cloned and the heteroduplex mobility assay can be performed on individual clones.

In other embodiments, the method comprises analyzing an HCV nucleic acid molecule, e.g., a reverse transcribed HCV plus or minus strand RNA molecule or fragment thereof, obtained from a cell, e.g., an induced cell, or viral particle, e.g., a viral particle produced by an induced cell, by restriction fragment length polymorphism (RFLP) analysis. In preferred embodiments, the HCV nucleic acid molecule is a fragment, e.g., a fragment comprising an HCV non-coding region (e.g., a 5′UTR or 3′UTR region) or an HCV protein coding region, e.g., a core, E1, E2, NS2, NS3, NS4a, NS4b, NS5a, or NS5b coding region. In some embodiments, the HCV nucleic acid molecule has been amplified, e.g., by RT-PCR. In preferred embodiments, RFLP analysis is performed on more than one HCV nucleic acid molecule. In particularly preferred embodiments, RFLP analysis is performed on at least 5, 10, 15, 20, or more individual HCV nucleic acid molecules. In preferred embodiments, RFLP analysis is performed on a plurality (e.g., thousands) of individual HCV nucleic acid molecules simultaneously, e.g., a single restriction digest reaction can be performed on a set of HCV nucleic acid molecules wherein the set contains more than one unique HCV nucleic acid sequence. In other embodiments, RFLP analysis is performed on two or more HCV nucleic acid molecules individually, e.g., the HCV nucleic acid molecules can be cloned and RFLP analysis can be performed on individual clones.

In another aspect, the invention features a method of screening for compounds (e.g., proteins, peptides, small molecules, or nucleic acids, such as inhibitory double-stranded ribonucleic acids (dsRNAi) or ribozymes) that alter the HCV mutation rate. The method includes (1) providing a test cell containing a nucleic acid of the invention, (2) inducing the inducible promoter of the nucleic acid, (3) contacting the test cell with a candidate compound, and (4) detecting a change in the number of HCV quasispecies produced by the cell in the presence of the candidate compound, as compared to the number of HCV quasispecies produced by a control cell, wherein said change in the number of HCV quasispecies produced indicates that the candidate compound alters the mutation rate of HCV.

In some embodiments, the candidate compound is a known antiviral agent, while in other embodiments, the candidate compound is not a known antiviral agent. In some embodiments, the candidate compound is a ribonucleoside analog, while in other embodiments, the candidate compounds is not a ribonucleoside analog.

In some embodiments, the detecting step includes the amplification, e.g., by RT-PCR, of HCV nucleic acid molecules, e.g., plus or minus strand HCV RNA molecules or fragments thereof, present in the cell or in viral particles produced by the cell. In preferred embodiments, the detecting step includes determining the sequences of HCV nucleic acid molecules, e.g., plus or minus strand HCV RNA molecules or fragments thereof, obtained from the cell or viral particles produced by the cell. In particularly preferred embodiments, the detecting step includes determining the sequences of at least 5, 10, 15, 20, or more individual HCV nucleic acid molecules. In other embodiments, the detecting step includes performing a heteroduplex mobility assay on a set of HCV nucleic acid molecules obtained from the cell or viral particles obtained from the cell. In some embodiments, the heteroduplex mobility assay is performed on the set of HCV nucleic acid molecules simultaneously (i.e., collectively), while in other embodiments the heteroduplex mobility assay is performed on each member of the set of HCV nucleic acid molecules individually. In still other embodiments, the detecting step includes performing RFLP analysis of a set of HCV nucleic acid molecules, e.g., reverse transcribed HCV nucleic acid molecules, obtained from the cell or viral particles produced by the cell.

In preferred embodiments, the change in the number of HCV quasispecies produced by the cell in the presence of the candidate compound, as compared to the number of HCV quasispecies produced by a control cell (i.e., in the absence of the candidate compound), is an increase, indicating that the compound increases the mutation rate of HCV. In other embodiments, the change in the number of HCV quasispecies produced by the cell in the presence of the candidate compound, as compared to the number of HCV quasispecies produced by a control cell, is a decrease, indicating that the compound decreases the mutation rate of HCV.

As used herein, a “change in the number of HCV quasispecies” refers to any quantitative or qualitative difference in the HCV nucleic acid molecules produced by a cell in the presence of a candidate compound, as compared to the HCV nucleic acid molecules produced in the control cells. For example, in a set of HCV nucleic acid molecules that have been sequenced, the number of base alterations divided by the number of nucleic acid molecules sequenced can be used as a quantitative statistic for comparing the set of HCV nucleic acid molecules produced by a cell in the presence of a candidate compound and the set of HCV nucleic acid molecules produced in the control cells. In a preferred embodiment, the change is statistically significant.

In some embodiments, the method of screening for compounds that increase the mutation rate of HCV further comprises passaging infectious HCV viral particles produced by an induced cell. In some embodiments, the infectious HCV viral particles are passaged one or more times. In some embodiments, the passaging occurs prior to detecting a change in the number of HCV quasispecies produced by a cell in the presence of the candidate compound, as compared to the number of HCV quasispecies produced by a control cell. In other embodiments, detecting a change in the number of HCV quasispecies produced by a cell in the presence of the candidate compound, as compared to the number of HCV quasispecies produced by a control cell, occurs at about the same time as passaging, e.g., HCV quasispecies can be identified in a subset of the infectious HCV viral particles being passaged or in the cells that produced the infectious HCV viral particles.

As used herein, an “infectious hepatitis C virus” or an “infectious HCV” means an HCV that is capable of propagation in a population of cells in vivo or in vitro. Therefore, an infectious hepatitis C virus minimally contains (1) a sequence encoding a precursor protein and (2) 5′ and 3′ non-translated flanking sequences sufficient to support virus replication (i.e., each step of the virus life cycle) in a cell population.

As used herein, an “HCV quasispecie” refers to an HCV nucleic acid molecule, such as a plus or minus strand HCV RNA molecule, or a viral particle that contains an HCV nucleic acid molecule, wherein the nucleic acid molecule has a sequence that differs from at least a portion of the infectious HCV sequence present in a nucleic acid molecule of the invention by one or more nucleic acid residues.

As used herein, one genetic element being “operably linked” to another means that a genetic element (either in a plus strand, minus strand, or double stranded form) is structurally configured to operate or affect another genetic element. For example, a promoter operably linked to a sequence encoding a polypeptide means that the promoter initiates transcription of a nucleic acid encoding the polypeptide, and a transcription termination signal operably linked to the sequence encoding the polypeptide means that the transcription termination signal terminates transcription of a nucleic acid encoding the polypeptide.

As used herein, an “error catastrophe” refers to a mutation rate of a virus, wherein the mutation rate is so high that essentially all of the viral progeny produced during viral replication are somehow defective, thereby resulting in a collapse in the viral population. In vivo, such a collapse would bring about the attenuation or end of the viral infection.

As used herein, a “test cell” is a cell that contains replicative HCV nucleic acid molecules, i.e., plus and minus strand HCV RNA molecules. Typically, the test cell is contacted with a candidate compound.

As used herein, a “control cell” is a cell that is the same cell type as “test cell”, and contains replicative HCV nucleic acid molecules, i.e., plus and minus strand HCV RNA molecules. Typically, the control cell is treated the same as the test cell, with the exception that it is not contacted with a candidate compound.

The nucleic acids and methods of the invention provide an in vitro HCV replication system capable of producing HCV quasispecies, thus allowing the mutation rate of HCV to be determined. Using this system, compounds having potential therapeutic value as anti-HCV agents can be quickly screened and identified based on their ability to increase the HCV mutation rate in vitro. Such compounds could function as anti-HCV agents by inducing an “error catastrophe” during HCV replication. Thus, the nucleic acids and methods of the invention provide a substantial benefit to anti-HCV drug development.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic diagram of the infectious HCV production system described herein. “δ” is the hepatitis D ribozyme. “T7” above the box, on the left side of the schematic diagram for “DNA” is the T7 promoter. “T7” above the box, on the right side of the schematic diagram for “DNA” is the T7 transcription termination signal. “vvT7” is the vaccinia virus vector encoding the T7 RNA polymerase.

FIG. 2 is a schematic diagram of a non-infectious control DNA construct and of the positive strand RNA produced from it.

FIG. 3 is a diagram summarizing the data presented in Table 4. The diagram shows the total number of mutations detected in all HCV genomic regions sequenced, including the 5′ UTR, core, E2, NS5A, and NS5b regions, as a function of the conditions of the in vitro HCV replication assay, such as the presence of ribavirin (RBV) at different doses and the presence of interferon-α (IFN). The T7 column represents the number of HCV mutations detected in HCV nucleic acid molecules produced by control cells. The data are further broken down into the categories of non-synonymous and synonymous mutations.

FIGS. 4A-F depict sequence alignments for replicating RNA sequences corresponding to selected HCV genomic regions under treated and untreated conditions. RNAs were harvested, reversed transcribed, cloned, and independently sequenced. FIG. 4A are alignments of cDNA sequences of E1/E2 HVR-1 and NS5B sequences following T7/full-length H77 transfection under untreated conditions. FIGS. 4B-D are alignments of 5′UTR, core, E1/E2 HVR-1, NS5A, and NS5B sequences under treatment with RBV at 50 μM (B) or 400 μM (C) or with IFN-α at 100,000U (D). FIGS. 4E and F are alignments of 5′UTR, core, and E1/E2 HVR-1 sequences following T7/BglII transfection under untreated conditions (E) or under treatment with RBV at 50 μM (F). Alignments were performed and compared with the parent H77 sequence.

Like reference symbols in the various drawings indicate like elements.

### DETAILED DESCRIPTION

A nucleic acid molecule of the invention, e.g., an expression plasmid, can be constructed using standard methods and reagents in the art of molecular biology. For example, inducible promoters, such as the T7 promoter system (Aoki et al., Virology 250:140-150, 1998; WO 98/39031) can be used to provide controlled expression of the infectious HCV clone. Alternatively, a tetracycline-inducible promoter system can be used (Moradpour et al., Hepatology 28:192-201, 1998). The inducible promoter is then operably linked to an infectious clone of an HCV, such as the one described in Yanagi et al., Proc. Natl. Acad. Sci. USA 94:8738-8743, 1997; or Kolykhalov et al., Science 277:570-574, 1997. At the 3′ end of the HCV clone, a ribozyme (e.g., a hepatitis D virus ribozyme) and optionally a transcription termination signal (e.g., a T7 transcription termination signal) are attached. Ideally, a cis-acting ribozyme, such as a HDV ribozyme, that cleaves at the boundary with the 3′ terminus of the HCV RNA is suitable for this purpose. After the HCV clone is transcribed, the ribozyme serves to remove itself and other 3′ sequences which may hinder virus replication.

Depending on the inducible promoter used to drive transcription of the infectious HCV clone, the method for inducing HCV transcription, and hence HCV replication, will vary. For example, if a T7 promoter is used, then transcription is induced by expressing a T7 RNA polymerase in a cell harboring the nucleic acid (e.g., as a result of transfection, viral vector delivery, or genomic DNA integration). The polymerase can be expressed using a viral vector, such as the vaccinia vector described in the Examples below, or the adenovirus vector described in Aoki et al., supra. Alternatively, a T7 polymerase expression vector under the control of a mammalian promoter can be introduced into the cell to induce HCV production. In addition, the cell can already contain a stably integrated expression cassette that is induced to express T7 polymerase, thereby producing replicative HCV.

Any cell that supports HCV replication, such as a CV-1 cell or a hepatic cell (e.g., HepG2), is suitable for use in the invention. Preferable cells include those cells which can be infected by infectious HCV viral particles and which subsequently support HCV replication.

To allow propagation (i.e., passaging) of HCV, induced cells are optionally mixed with a population of cells that lack replicative HCV, e.g., plus or minus strand HCV RNA, but are permissive for HCV infection. The cells that lack replicative HCV can either contain or not contain a nucleic acid of the invention. A mixed or unmixed population of cells can be used in screening assays for candidate antiviral compounds.

After the initiation of HCV replication in one or more cells, HCV RNA, e.g., plus or minus strand HCV RNA, can be isolated, e.g., from the cells or from viral particles produced by the cells, for the purposes of identifying and/or quantifying the number of HCV quasispecies produced. The identification and/or quantification of the number of HCV quasispecies produced in the in vitro HCV replication system can be performed using cells that have been contacted with a candidate compound, as well as cells that have not been contacted with a candidate compound.

The identification of HCV quasispecies can be performed by any suitable direct or indirect method. For example, the HCV RNA can be reverse transcribed and amplified by PCR, and the resulting PCR product can be sequenced. The sequence of individual nucleic acid molecules present in the PCR product can be determined, e.g., by subcloning the PCR product and sequencing one or more independent subclones. Preferably, at least 5, 10, 15, 20, or more individual HCV nucleic acid molecules are sequenced. Alternatively, the PCR product could be sequenced directly. Direct sequencing would not indicate the sequence of individual HCV nucleic acid molecules, but could provide information about locations where subsets of HCV nucleic acid molecules differ in their sequence. The method of sequencing can be any known to the art, e.g., the di-deoxy chain termination method of sequence determination. The method of sequencing can also be a technique currently being developed, e.g., involving microarray hybridization or the passage of DNA or RNA through a pore, wherein the conductance of the pore provides an indication of the base that is passing through. If possible, the sequence of quasispecie HCV nucleic acid molecules is determined without first amplifying the HCV nucleic acid molecules, e.g., without first producing an RT-PCR product. The generation of HCV quasispecies can be quantified, e.g., as the number of nucleic acid changes in a particular length of sequence relative to a standard sequence, e.g., an infectious HCV nucleic acid sequence present in a nucleic acid molecule of the invention or fragment thereof, divided by the total number of nucleic acid molecules sequenced.

Techniques that provide an estimate of the number of quasispecie HCV nucleic acid molecules produced in the in vitro HCV replication system are also suitable for use in the methods of the invention. For example, the HCV RNA can be analyzed using a heteroduplex mobility assay. Hassoba et al. (1999), J. Hepatology 31:618-27, have shown that there is a linear correlation between the mobility of heteroduplexes and the number of nucleotide substitutions present in the nucleic acid molecule being analyzed. The method of Hassoba et al. (1999), supra, relies upon the analysis of individual nucleic acid molecules that have been cloned. However, the method could potentially be adapted to the simultaneous analysis of a set of nucleic acid molecules, by observing the mobility distribution of the set of heteroduplexes; differences in the mobility distribution of heteroduplexes generated from the nucleic acid molecules of a test cell (i.e., the cell that is exposed to a candidate compound) vs. a control cell could provide a qualitative means of detecting a change in the amount of HCV quasispecies produced in the test cell.

Alternatively, the HCV RNA can be amplified, e.g., by RT-PCR, and the resulting PCR product can be directly analyzed by restriction fragment length polymorphism (RFLP) analysis. To avoid the amplification of the HCV RNA, it can be converted into cDNA and then analyzed by RFLP analysis. Using such a technique, it may be necessary to visualize the RFLP products using a probe, e.g., a fluorescently or radioactively labeled nucleic acid molecule. An alternate way to avoid amplification of the HCV RNA is to analyze it by RNase protection, which also requires the use of a probe, e.g., a fluorescently or radioactively labeled nucleic acid molecule.

In general, RFLP analysis is performed on a set of nucleic acid molecules, wherein the set contains subsets of nucleic acid molecules that have a particular sequence but differ from one another by at least one nucleic acid residue. RFLP analysis involves digesting the set of nucleic acid molecules with one or more restriction endonucleases (usually the digestions are performed in parallel) and analyzing the length and abundance of nucleic acid fragments resulting from the digestion. Differences between samples can be observed. For example, when the fragments are separated by size, e.g., on a polyacrylamide or agarose gel, differences in the amount of one fragment, or in the appearance of new bands or the disappearance of bands, maybe detected. Such differences provide qualitative information. Quantification of the amount of nucleic acid molecules initially digested and the resulting fragments can provide more quantitative information.

The identification of HCV quasispecies can be performed using full length HCV RNA molecules, e.g., plus or minus strand RNA molecules, or fragments thereof. A fragment of an HCV RNA molecule can include an HCV non-coding region, e.g., a 5′UTR or 3′UTR region, or an HCV protein coding region, e.g., a core, E1, E2, NS2, NS3, NS4a, NS4b, NS5a, or NS5b coding region. However, the fragments are not restricted to regions that have a defined function, such as those that encode a particular protein. Rather, the fragments can include any region, including those regions that are particularly amenable to analysis.

For the purposes of identifying compounds that increase the mutation rate of HCV, the identification of HCV quasispecies can be performed after contacting an induced cell, or a cell infected with an infectious HCV viral particle, e.g., during passaging, with a candidate compound. HCV replication can be induced in the induced cell either before or after the induced cell is contacted with the candidate compound. In general, the cell is induced about twenty hours after contacting the cell with the candidate compound, and the HCV RNA is isolated about twenty-four hours after induction. These times can be adjusted as appropriate and should not be construed as invariant aspects of the methods of the invention. The identification of compounds that increase the mutation rate of HCV necessarily involves identifying HCV quasispecies produced in both a test cell and a control cell, though the control need not be repeated for every test. The test cell is contacted with the candidate compound while the control cell is not. Otherwise, the test cell and control cell are of the same cell type and are treated and analyzed identically. Differences in the number of HCV quasispecies produced in the test cell and control cell can be determined qualitatively or quantitatively by any of the methods discussed herein. Preferably, the differences are quantitative and statistically significant. There are many different ways of measuring statistical significance, including, e.g., the X² test, all of which are within the scope of the invention.

Depending on the nature of a candidate compound, contacting the cell with the compound can involve a variety of standard techniques. If the compound is active from outside the cell, all that is needed for the contacting step is the addition of the compound to the aqueous mixture containing the cells. Similarly, if the candidate compound is a small molecule drug which is active within the cell and which can pass through lipid membranes, all that is needed for the contacting step is the addition of the compound to an aqueous mixture containing the cells. However, if the compound is a protein or nucleic acid intended to elicit its antiviral effect inside a cell, the contacting step may require other techniques. A protein compound can be delivered into the cell by encapsulation in liposomes or as a fusion to a viral protein that is delivered inside a cell by viral infection. A nucleic acid, e.g., double stranded inhibitory RNA (i.e., dsRNAi, preferably 21 base pairs in length) or a ribozyme, can be delivered into the cell using a viral vector or a transfection protocol (e.g., electroporation). Methods of introducing proteins and nucleic acids into a cell are well known in the art of molecular biology.

Compounds that can be screened by the methods described herein, i.e., candidate compounds, include known antiviral agents as well as compounds that are not known to have antiviral activity. Known antiviral agents include, but are not limited to, ABACAVIR, ABACAVIR/LAMIVUDINE/ZIDOVUDINE, ACYCLOVIR, ADEFOVIR, ALDESLEUKIN, AMANTADINE, AMPRENAVIR, ATEVIRDINE, BRIVUDINE, CIDOFOVIR, DELAVIRDINE, DIDANOSINE, DOCOSANOL, EFAVIRENZ, EMIVIRINE, EMTRICITABINE, FAMCICLOVIR, FOMIVIRSEN, FOSCARNET, GANCICLOVIR, IDOXURIDINE, INDINAVIR, INTERFERON ALFA-2A, INTERFERON ALFA-2B, INTERFERON ALFA-N1, INTERFERON ALFA-N3, INTERFERON ALFACON-1, INTERFERON BETA (natural), INTERFERON BETA-1A, INTERFERON BETA-1B, INTERFERON GAMMA, ISOPRINOSINE, LAMIVUDINE, LAMIVUDINE/ZIDOVUDINE, LOPINAVIR/RITONAVIR, LOVIRIDE, NELFINAVIR, NEVIRAPINE, OSELTAMIVIR, PEGINTERFERON ALFA-2B, PENCICLOVIR, PENTAFUSIDE, PLECONARIL, RIBAVIRIN, RIMANTADINE, RITONAVIR, SAQUINAVIR, SORIVUDINE, STAVUDINE, TECELEUKIN, TENOFOVIR, TIPRANAVIR, TRIFLURIDINE, VALACYCLOVIR, VALGANCICLOVIR, VIDARABINE, ZALCITABINE, ZANAMIVIR, and ZIDOVUDINE, as well as 1-isobutyl-1H-imidazo[4,5-c]quinolin-4-amine, 1-(2-hydroxy-2-methylpropyl)-1H-imidazo[4,5-c]quinoline-4-amine, and other compounds disclosed in U.S. Pat. No. 4,689,338, the contents of which are incorporated herein by reference. Typically one would screen compounds of the same general class as, or structurally similar to, any of the above-listed drugs.

Compounds that can be screened by the methods described herein, i.e., candidate compounds, include ribonucleoside analogs as well as compounds that are not ribonucleoside analogs. Known ribonucleoside analogs include but are not limited to N⁴-aminocytidine, N¹-methyl-N⁴-aminocytidine, 3, N⁴-ethenocytidine, 3-methylcytidine, 5-hydroxycytidine, N⁴-dimethylcytidine, 5-(2-hydroxyethyl) cytidine, 5-chlorocytidine, 5-bromocytidine, N⁴-methyl-N⁴-aminocytidine, 5-aminocytidine, 5-nitrosocytidine, 5-(hydroxyalkyl)-cytidine, 5-(thioalkyl)-cytidine and cytidine glycol. A class of preferred uridine analogs includes 5-hydroxyuridine, 3-hydroxyethyluridine, 3-methyluridine, O²-methyluridine, O²-ethyluridine, 5-aminouridine, O⁴-methyluridine, O⁴-ethyluridine, O⁴-isobutyluridine, O⁴-alkyluridine, 5-nitrosouridine, 5-(hydroxyalkyl)-uridine, and 5-(thioalkyl)-uridine. A class of preferred adenosine analogs includes 1, N⁶-ethenoadenosine, 3-methyladenosine, and N⁶-methyladenosine. A class of preferred guanosine analogs includes 8-hydroxyguanosine, O⁶-methylguanosine, O⁶-ethylguanosine, O⁶-isopropylguanosine, 3, N²-ethenoguanosine, O⁶-alkylguanosine, 8-oxo-guanosine, 2, N³-ethenoguanosine, and 8-aminoguanosine.

The methods of the invention can be performed repetitively and in parallel to screen libraries of compounds (e.g., a small molecule library, a peptide library, a single chain antibody library, or a dsRNAi library) for candidate drugs. For example, cells having a nucleic acid of the invention can be cultured in the wells of a 96-well microtiter plate. Before or after induction of HCV replication, each member of the compound library is deposited into a well, and after a suitable amount of time the mutation rate of HCV is determined for the well. Similarly, this arrangement can be adapted to allow for passaging of infectious HCV viral particles, e.g., by transfering cell culture medium from a first well to a second well after a sufficient amount of time has passed to allow infectious HCV viral particles to be produced by the cells in the first well. Automation of the methods of the invention is especially amenable to high throughput screening of chemical libraries.

One skilled in the art can, based on the above disclosure and the example described below, utilize the present invention to its fullest extent. It will be understood that various modifications may be made without departing from the spirit and scope of the invention. The following examples are to be construed as merely illustrative of how one skilled in the art can make and use the inducible HCV replication system described herein. Other embodiments are within the scope of the following claims. Any publications cited in this disclosure are incorporated in their entirety by reference.

### EXAMPLES

### Example 1

Preparation of Constructs

The HCV construct originally used to generate infectious transcripts was the H77 clone (Yanagi et al., Proc. Natl. Acad. Sci. USA 94:8738-8743, 1997). The plasmid containing the clone, pCV-H77, contains the full-length genotype 1a HCV sequence of strain H77. The plasmid contains a T7 promoter immediately upstream of the viral cDNA and was adapted at its immediate 3′ terminus with the hepatitis delta virus cis-acting ribozyme (Wang et al., Nature 323:508-514, 1986) in continuity with the T7 terminator sequence. To accomplish this, a synthetic antisense oligonucleotide was made, the oligonucleotide being the complement of the following contiguous sequences: the terminal 3′ 20 nucleotides of the H77 strain, followed immediately by the 85-nucleotide HDV ribozyme, followed immediately by the 48-nucleotide T7 terminator. This 153-nucleotide sequence was divided into two overlapping 85-nucleotide primers, each sharing 17 nucleotides in common. These primers were synthesized (IDT, Coralville, Iowa) and used as the downstream primers in a series of PCR reactions.

A sense oligonucleotide of 85 nucleotides in length, corresponding to HCV H77 sequences 9367-9451, was also synthesized in a similar manner. H77 numberings, as use herein, are as designated in Yanagi et al., supra. The sense primer and the inner antisense primer were used in a PCR reaction with 10 ng of pCV-H77 as a template under the following conditions: 50 pmol of primer, 0.5 U Taq polymerase, 1.5 mM MgCl2, 0.5 mM each dNTP. The 100 ml reaction was carried out with 20 cycles of PCR under the following cycling conditions: 95° C. for 1 minute, 65° C. for 1 minute, and 72° C. for 1 minute. The PCR product was gel purified (QIAquick, QIAgen, Chatsworth, Calif.) and cloned into the vector pcDNA3.1/V5/His-TOPO (Invitrogen, Carlsbad, Calif.). This product then served as the template for a second PCR reaction under identical conditions using the sense primer and the outer antisense primer. The product of the second amplification was again cloned into pcDNA3.1/V5/His-TOPO to generate pHCV-Rz-TOPO. The sequence of this product was confirmed bi-directionally by dideoxy sequencing.

Because the parent plasmid for pCV-H77C, pGEM-9z, contained a second T7 promoter at its original multiple cloning site just downstream of the HCV cDNA, this sequence was removed by excising the XbaI-SfiI fragment. A synthetic XbaI-MlUI-MluI-SfiI linker pair was generated, and ligated into the XbaI-SfiI-digested pCV-H77C. Successful insertion of a single linker was confirmed by sequencing. This product, pCV-H77C-Mlu, was digested with AflII and MluI, and the AflII-MluI fragment isolated from pHCV-Rz-TOPO was subcloned into this vector, to yield pT7-flHCV-Rz. Successful insertion of HDV Rz and T7 terminator sequences was confirmed by bi-directional sequencing.

To confirm the dependence of the replication system on intact HCV non-structural proteins, which had been postulated to be essential for RNA replication, a deletion mutant was produced by removing the BglII-BglII fragment from pT7-flHCV-Rz (FIG. 2). The deletion spanned HCV nucleotides 3237-8939. This deletion removed the downstream portion of NS2 to NS5B, inclusive of the active site motif of the NS5B RNA-dependent RNA polymerase. The 5.7 kb deletion was predicted to keep the polyprotein in frame. The deletion was confirmed by sequencing of pT7-HCVD BglII-Rz. For experiments designed to examine vaccinia-T7 efficiency, the positive control plasmid OS8 contained a T7 promoter flanking the β-galactosidase gene.

### Example 2

Cell Lines

Cells were maintained in DMEM (BRL Gibco, Rockville, Md.) containing penicillin (50 IU/mL) and streptomycin (50 mg/mL) and was supplemented with 10% fetal calf serum. CV-1 cells were obtained from the American Type Culture Collection. For some experiments, HepG2 and Huh7 cell lines were used for transfection/infection.

### Example 3

Transfection/Infection Experiments

Expression of HCV was carried out using an adaptation of the binary expression system described in Fuerst et al., Proc. Natl. Acad. Sci. USA 83:8122-8126, 1986. Subconfluent CV-1 cells, chosen because of their enhanced transfection efficiency, were transfected in 6 cm tissue culture plates using 1 mg of plasmid (pT7-flHCV-Rz, pT7-HCVDBglII-Rz, OS8) with 3 ml of Lipofectamine transfection reagent (GIBCO BRL) in serum-free DMEM. Following 4 hours of transfection, the cells were washed and replaced with DMEM containing 10% FCS. Twenty-four hours post-transfection, recombinant vaccinia-T7 polymerase (vTF7-3; Fuerst et al., supra) was added to the cells at an MOI of 10. Twenty-four hours post-vTF7-3 infection, cells were lysed and analyzed for RNA and protein expression. The positive control vector OS8 verified successful T7 polymerase expression.

### Example 4

Strand-Specific RT-PCR

To confirm successful HCV RNA strand synthesis, strand-specific RT-PCR was carried out on lysates of transfected/infected cells. RNA was extracted using TRIzol reagent (GIBCO BRL), and subjected to two rounds of DNase I digestion (5 U, Boehringer Mannheim, Indianapolis, Ind.) at 37° C. for 60 minutes. The RNA was then phenol/chloroform-extracted and resuspended in DEPC-treated water. The purified RNA was then subjected to strand-specific RT-PCR using primers corresponding to the 5′ and 3′ genomic and antigenomic H77 RNA. The following oligonucleotide sequences, all 20-mers, were used: 5′ end sense, H77 nt 29-48; 5′ end antisense, nt 390-371; 3′ end sense, nt 9241-9260; and 3′ end antisense, nt 9361-9342. For detection of genomic (+) strand RNA, the antisense primer was used for the reverse transcription step. For detection of antigenomic (−) strand RNA, the sense primer was used for the reverse transcription step. Reverse transcription was carried out in 20 ml reactions with the following components: 1 ml RNA, 25 pmol RT primer, 0.5 U AMV reverse transcriptase (Perkin Elmer, Branchburg, N.J.), 1.5 mM MgCl², 0.5 mM each dNTP, and 1U RNasin (Perkin Elmer). The reaction was carried out at 42° C. for 15 minutes, and the enzyme was heat inactivated for 10 minutes at 99° C. The resulting product was treated with three successive rounds of DNase I (Boehringer Mannheim) at 37° C. for 30 minutes each, and the final product was purified by phenol/chloroform extraction. The cDNA was then subjected to 25 cycles of PCR using 25 pmol each of the relevant sense and antisense primers, 0.5 mM each dNTP, 1.5 mM MgCl₂, and 0.5 U Taq™ polymerase. Reaction products were then analyzed by 1.6% agarose gel electrophoresis.

### Example 5

Ribonuclease Protection Assay (RPA)

To confirm HCV replicative RNA synthesis by a second line of investigation, RPA was performed on the extracted RNAs. A probe was used for specific detection of antigenomic HCV RNA at the 3′ terminus of the genome. To accomplish this, the vector pHCV-3′T (Chung et al., Biochem. Biophys. Res. Commun. 254:351-362, 1999) was used to generate a sense probe corresponding to the terminal HCV RNA. This vector contains the highly conserved 98-nucleotide 3′ terminal sequence (also conserved in H77) that had been adapted and cloned into the EcoRI and XbaI sites of pSP72. Following linearization of pHCV-3′T by XbaI, an a-32P-UTP-labeled probe was generated by in vitro transcription using T7 polymerase according to manufacturer's protocol (Ambion, Houston, Tex.). This probe was purified by phenol/chloroform extraction and then used for RPA. For all RPAs, the RPA II kit was used in accordance to manufacturer's instructions (Ambion). RPA products were separated by 8 M urea/5% PAGE. For the HCV replicative strand, the expected protected fragment size was 210 nucleotides. As a positive control, hybridization was performed with the pT7-flHCV-Rz DNA.

To generate RPA probes for human β-actin and GAPDH, commercially available antisense control DNA templates pTRI-β-actin and pTRI-GAPDH (Ambion) were used. As with the HCV template, in vitro transcription in the presence of a-32P-UTP was carried out using T7 RNA polymerase (MAXIscript, Ambion). The probes were phenol/chloroform extracted, purified, and used in RPA experiments conducted according to the manufacturer's instructions (RPA II, Ambion). To generate an antisense RPA probe for the detection of β galactosidase mRNA, a PCR approach was used in which the antisense primer was adapted upstream with the T7 promoter sequence. Oligonucleotide primers corresponding to the coding sequence of β-galactosidase in the vector OS8 were synthesized as follows. The sense strand was 5′-CCGTCGTTTTACAACGTCGTGACTGGGAAAACCCTG-3′ (SEQ ID NO:1), and the antisense was 5′-TATACGACTCACTATAGGCCATTCGCCATTCAGG-3′ (SEQ ID NO:2).

PCR was carried out using 10 ng of OS8 as template under the following conditions: 25 pmol each primer, 0.5 mM each dNTP, 1.5 mM MgCl², 0.5 U Taq polymerase. Twenty-five cycles were performed under the following conditions: 95° C. for 1 minute, 45° C. for 1 minute, 72° C. for 1 minute. PCR products were separated by agarose gel electrophoresis and gel purified (QIAquick, QIAgen). The purified template DNA was then used for generation of α-³²P-UTP-labeled antisense probe by in vitro transcription with T7 polymerase (MAXIscript, Ambion). RPA was carried out as described above. For β-actin, GAPDH, and β-galactosidase probes, hybridization was carried out using the original template DNAs as positive controls. For the ribavirin and amantadine studies, a simultaneous RPA using GAPDH probe with HCV (−) strand probe was performed, and the results displayed on the same gel.

### Example 6

Western Immunoblotting

Lysates were extracted using Laemmli 2× SDS sample-extraction buffer (Sigma, St. Louis, Mo.). Total protein was quantitated by the Bradford assay. Equal quantities of protein were loaded and separated by 10% SDS-PAGE. Following transfer of the gels to PVDF membranes, Western immunoblotting was carried out using the ECL-Western detection method (Amersham, Piscataway, N.J.). For HCV protein detection, polyclonal antisera were used. The antisera were pooled from HCV-infected patients whose sera were reactive by the recombinant immunoblot assay (RIBA-2, Abbott Labs, Chicago, Ill.) at a dilution of 1:50. Horseradish peroxidase-conjugated rabbit anti-human antibody (Amersham) was used at 1:5000 for detection. Five micrograms of recombinant HCV core protein (Austral Biologicals, San Ramon, Calif.) was used as a positive control. For β-galactosidase detection, monoclonal anti-β-galactosidase (Promega, Madison, Wis.) was used as the primary antibody at a dilution of 1:5000. For β-actin, monoclonal anti-actin antibody (Chemicon, Temecula, Calif.) was used at a dilution of 1:200. For both actin and galactosidase immunoblots, conjugated rabbit anti-mouse (Amersham) antibody at 1:10,000 dilution was used as secondary antibody.

### Example 7

Antiviral Inhibitor Studies

Purified recombinant IFN-α-2b (INTRON A, Schering Plough Research Institute, Kenilworth, N.J.) was used at incremental doses of 0, 2000, 6000, or 15,000 units/plate. The drug was added to the medium at the conclusion of transfection. Time course experiments indicated that IFN-α-2b's inhibition of vaccinia-T7 polymerase was minimized when IFN-α-2b was given 20 hours prior to the introduction of vTF7-3.

Ribavirin (Schering Plough Research Institute) doses of 0, 10, 20, 80, 120, 160, and 200 mg/plate were added to the medium 20 hours prior to introduction of vTF7-3. These doses were comparable to those achievable at physiologic concentrations (Ilyin et al., Hepatology 27:1687-1694, 1998). The results were unchanged when ribavirin was given 0 and 12 hours before vTF7-3 infection.

Amantadine-HCl (Sigma) was used at doses comparable to pharmacologically achievable concentrations (Shannon et al., Antimicrob. Agents Chemother. 20:769-776, 1981). Doses of 0, 1, 10, and 100 mg/plate were added to the medium at 20 hours prior to vTF7-3 infection. Results seen were unchanged when amantadine was given 0 and 12 hours before vTF7-3 infection.

### Example 8

Generation of a Binary HCV Expression System in Mammalian Cells

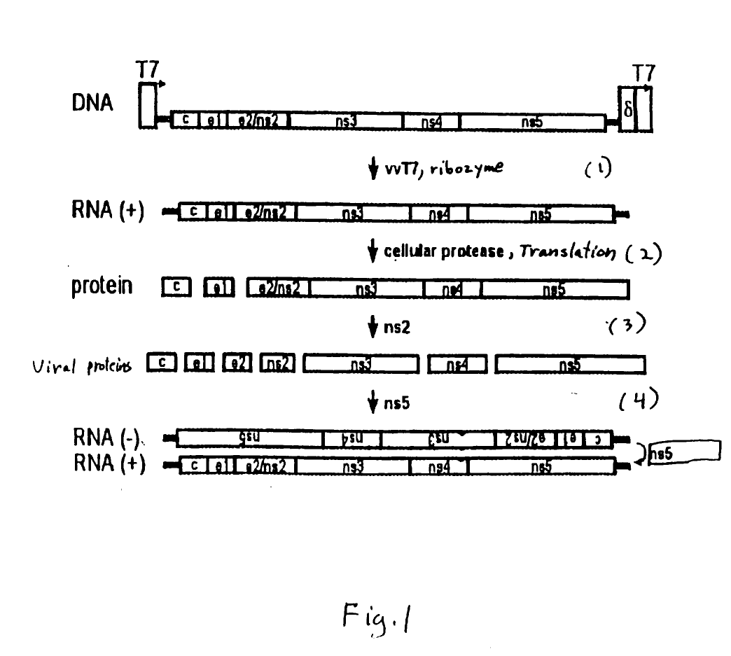

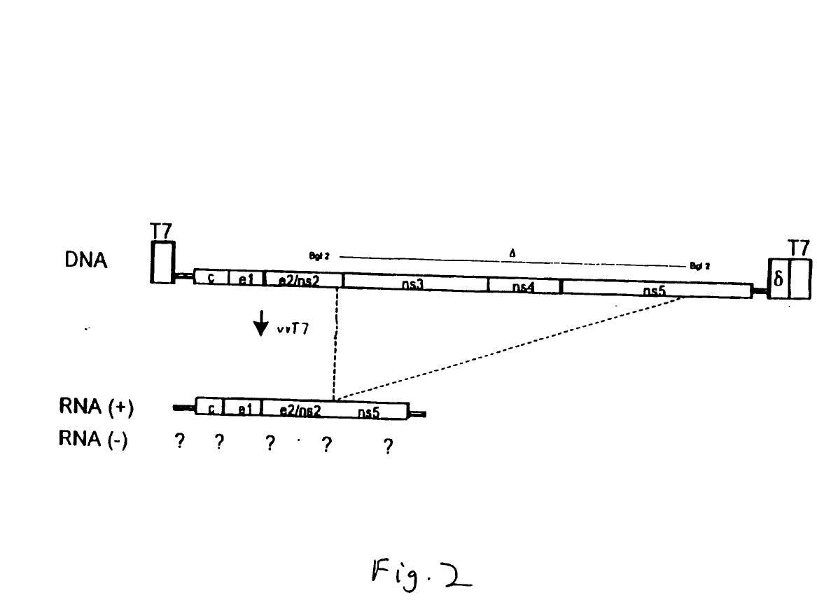

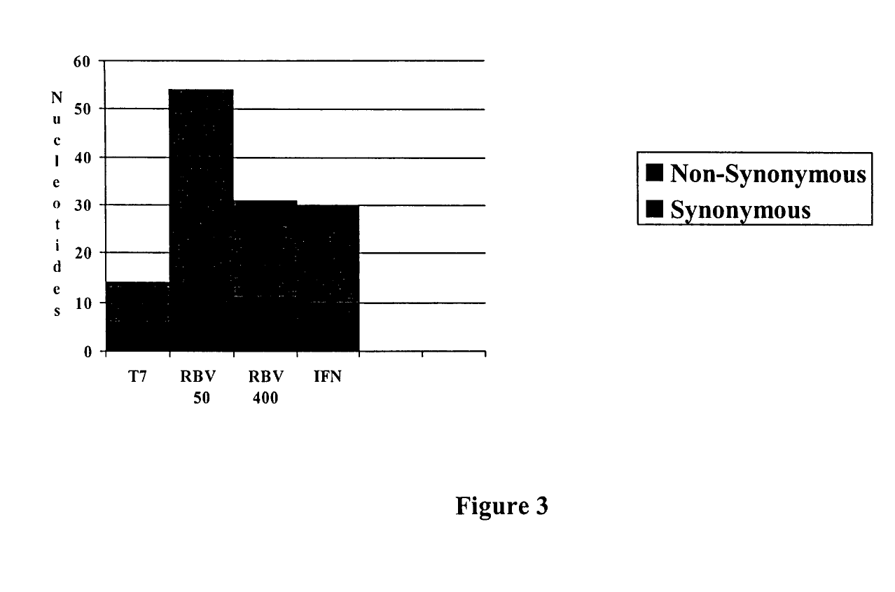

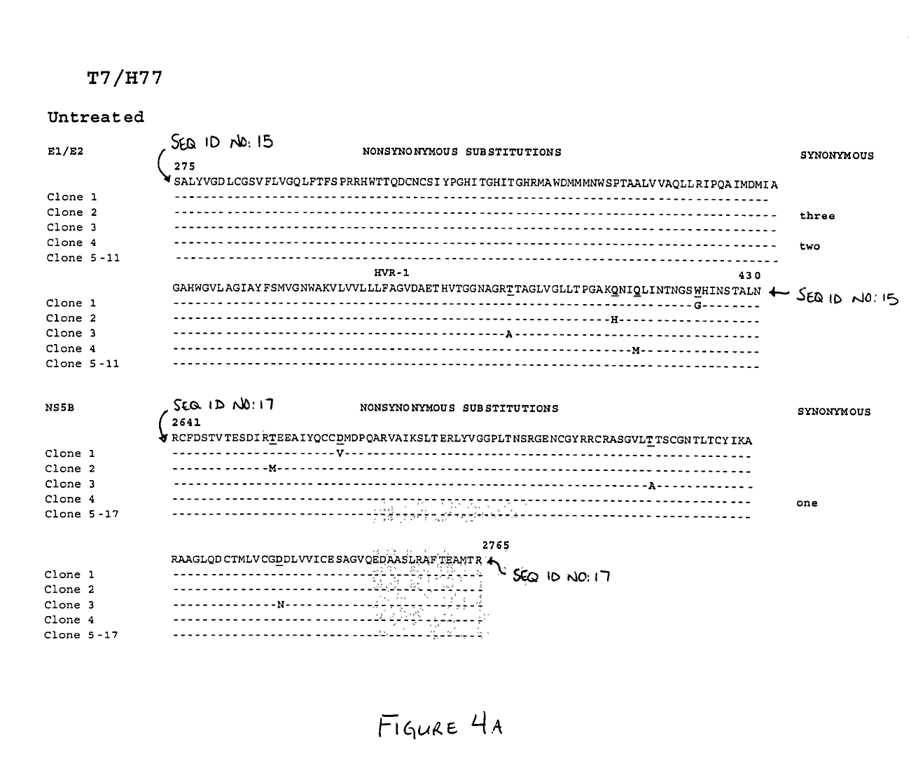

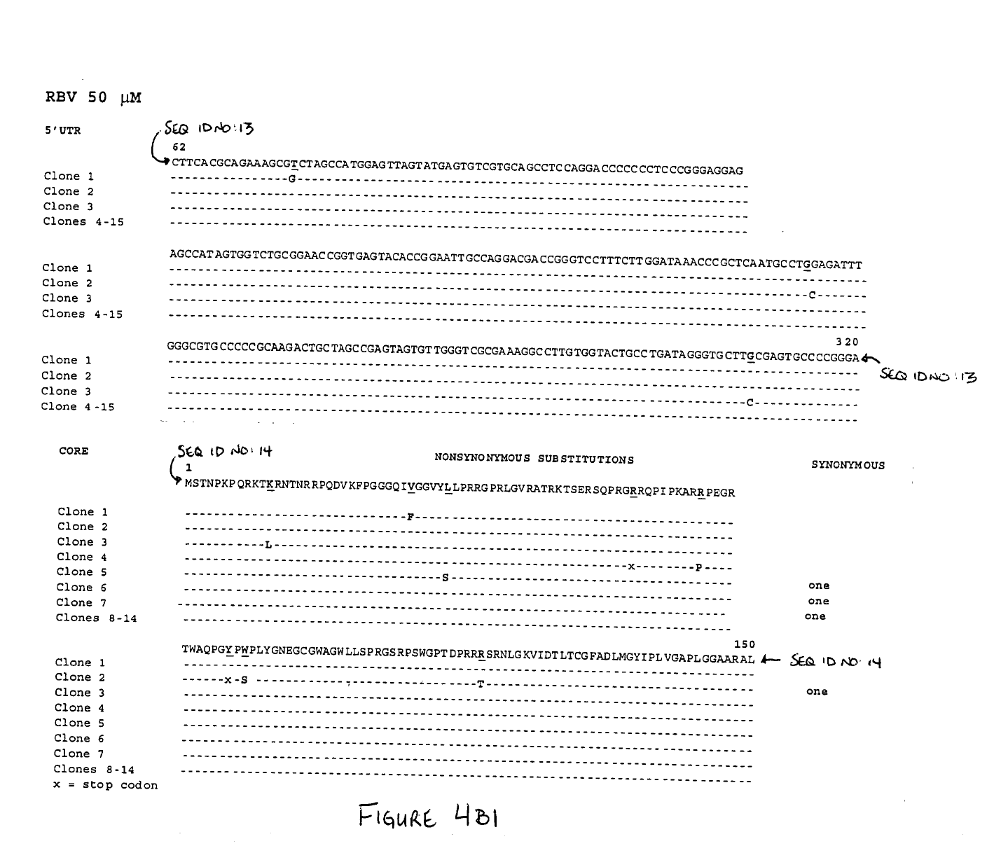

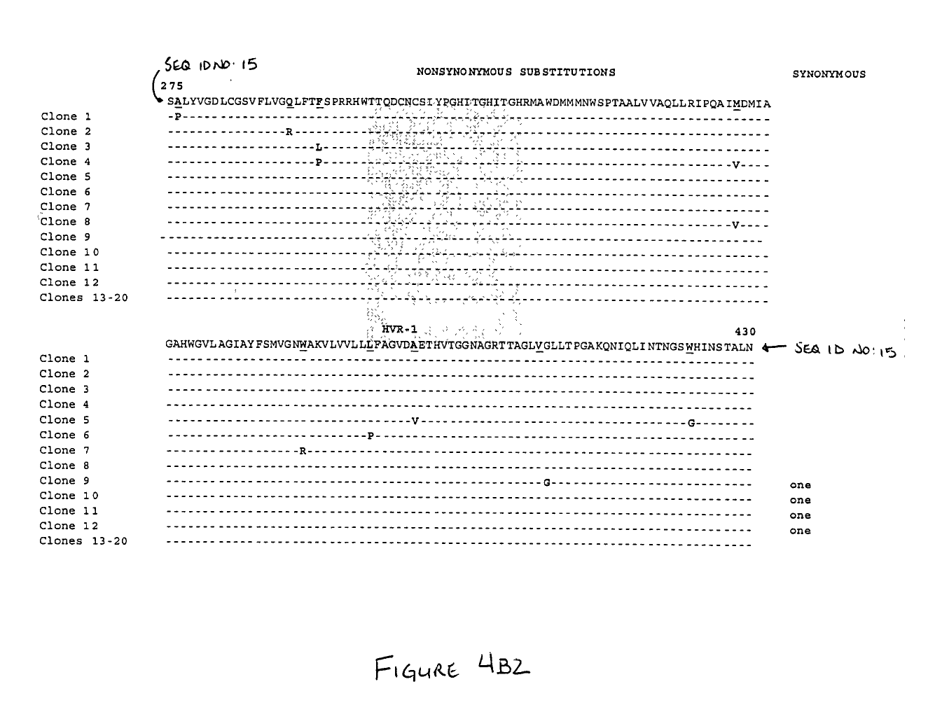

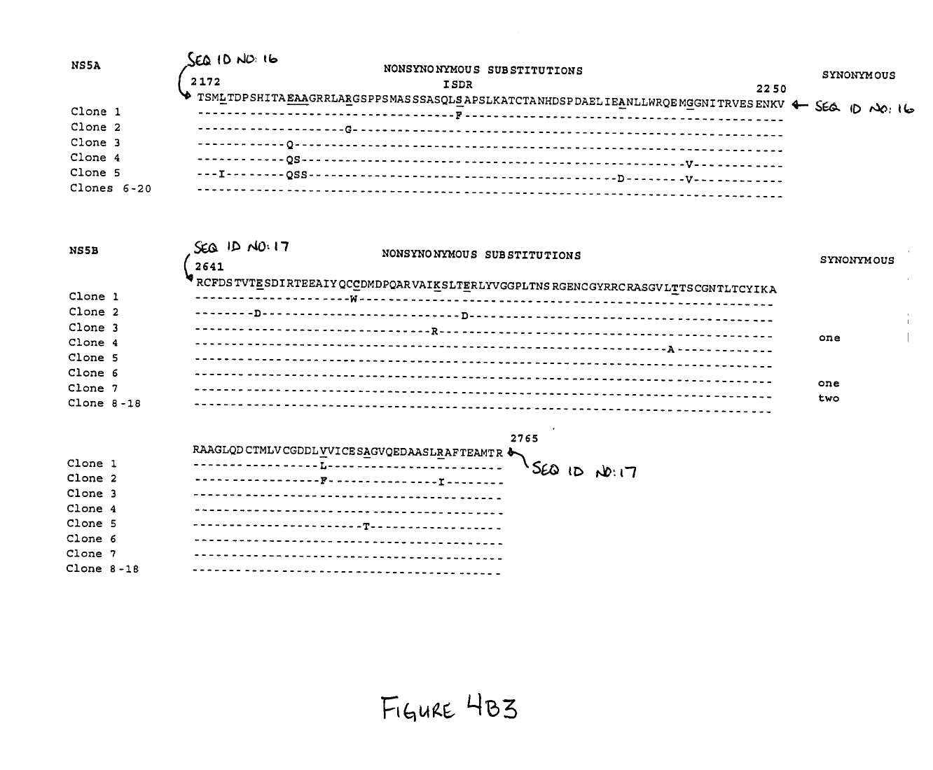

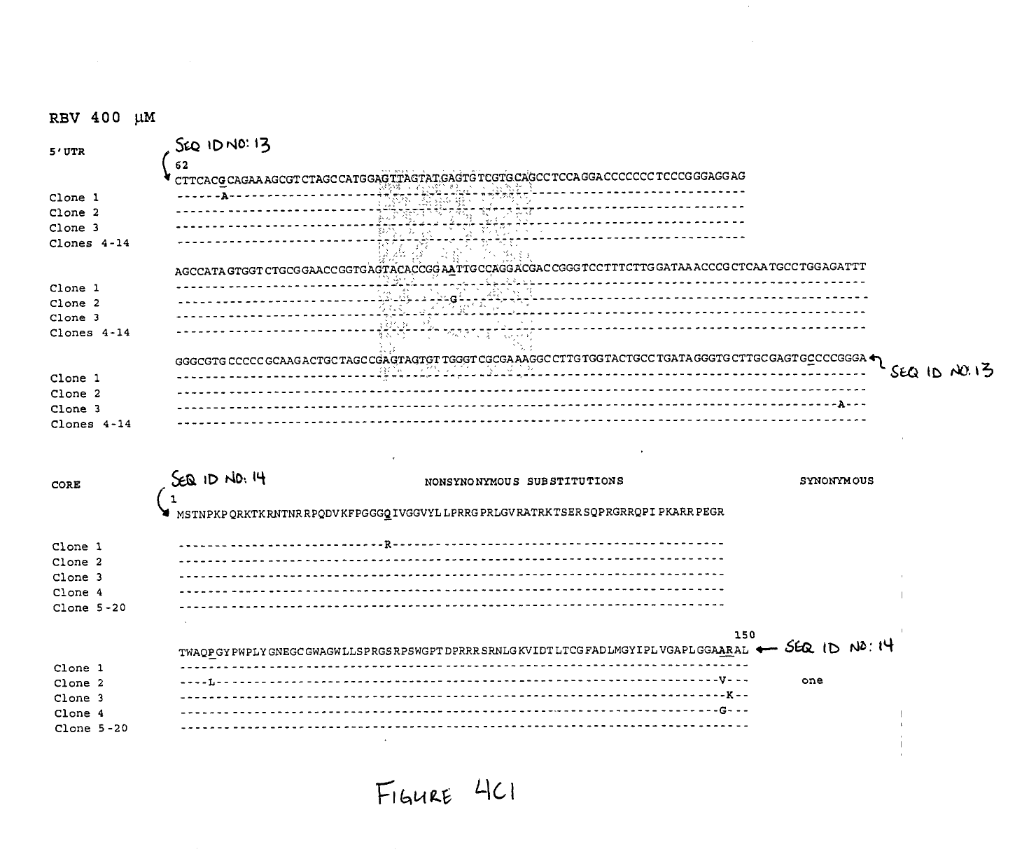

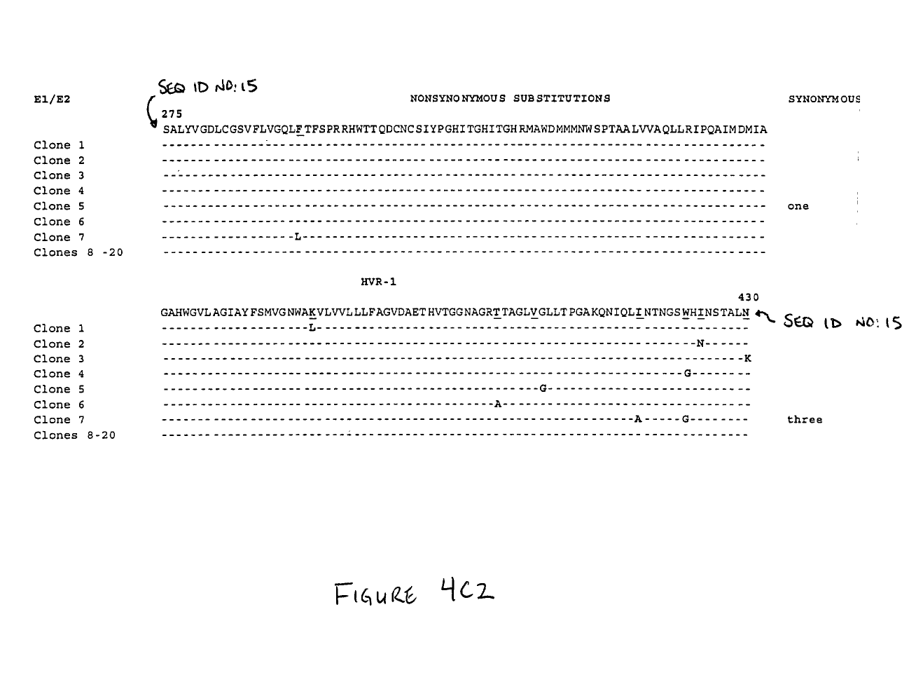

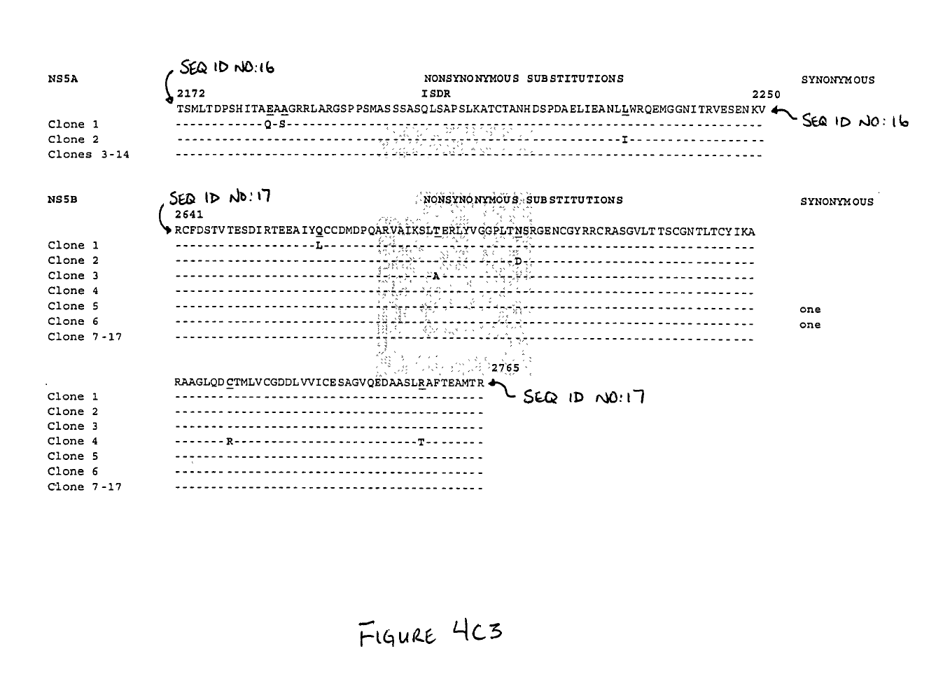

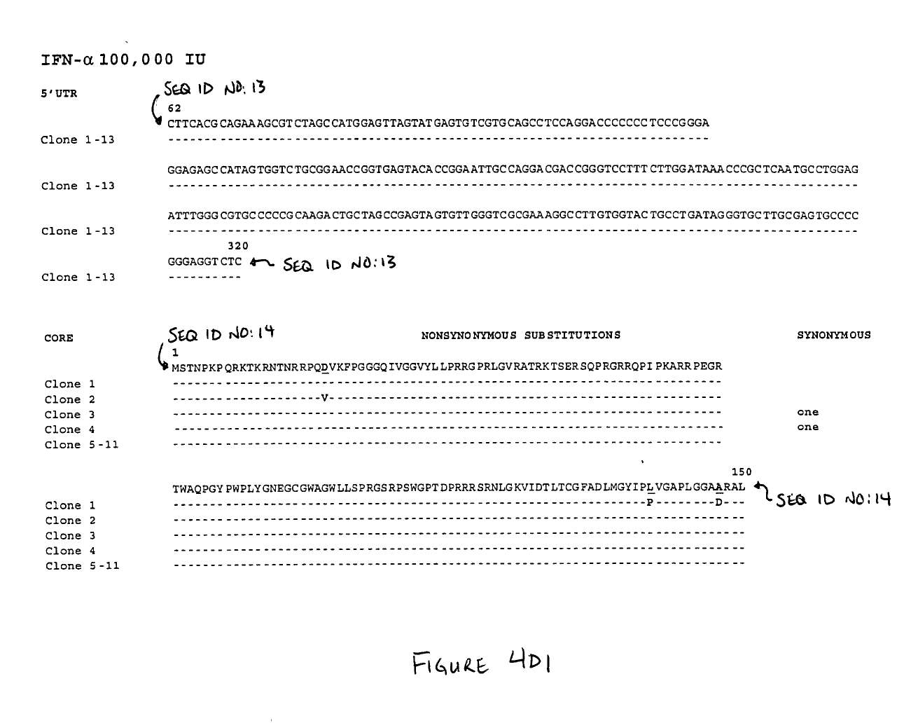

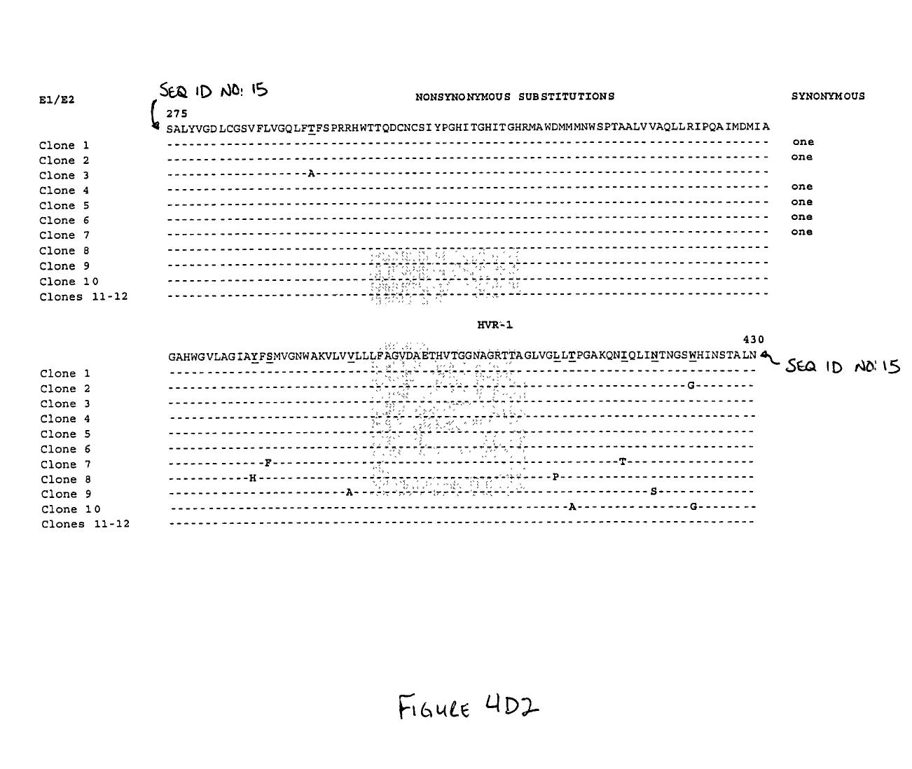

To explore whether the early steps in the HCV lifecycle could be recapitulated using a DNA-based approach, the plasmid pCV-H77 provided a starting point for the genetic manipulations. pCV-H77 contained a full-length genotype 1a sequence, inclusive of the highly conserved 5′ and 3′ untranslated sequences. pCV-H77 was adapted at its 3′ terminus with the cis-acting hepatitis delta ribozyme followed immediately by the T7 transcription termination sequence (FIG. 1). The final construct was designated pT7-flHCV-Rz. Because the upstream end of the viral sequence lies immediately downstream of the T7 promoter, T7 polymerase was expected to generate, by transcription, a full-length viral RNA genome bearing bona fide termini for strand replication.

When CV-1 cells were transfected with pT7-flHCV-Rz and infected with recombinant vaccinia encoding T7 polymerase (vTF7-3), detectable RNA corresponding to the genomic or (+) strand and the antigenomic or (−) strand of the viral genome was detected. Strand-specific RNA synthesis was detected by two lines of investigation: strand-specific RT-PCR and ribonuclease protection assay. As confirmation of full antigenomic strand synthesis, (−) strand RNA sequences corresponding to both the 5′ and 3′ untranslated portions of the genome (i.e., the 5′ and 3′ termini of the antigenomic strand) were detected by RT-PCR. Using an RNase protection assay, (−) strand RNA corresponding to the 3′ terminus of the genome was also detected. This result was dependent on transfection with HCV sequences (pT7flHCVRz) and infection with vTF7-3, but not observed with pT7-flHCV-Rz alone or on vTF7-3 combined with an unrelated template. Thus, the replication system succeeded in producing the full complement of viral protein synthesis.

To confirm successful viral protein synthesis, Western blotting was performed using polyclonal antisera directed against genotype 1 HCV. Immunoreactive HCV core protein was expressed in the presence of T7-flHCV-Rz and vaccinia-T7, but not with either alone or in the presence of vTF7-3 and an unrelated expression construct. The successful synthesis of β-galactosidase from a control vector confirmed the validity of our binary expression system for CV-1 cells.

HCV antigenomic strand synthesis is dependent on full-length HCV sequences. To confirm that this specific RNA strand synthesis was dependent on the expression of viral proteins, as expected, the experiment described above was repeated with an expression construct deleted in the region from NS2-NS5 (FIG. 2). This deleted region contained the NS3 RNA helicase and the key catalytic domains of the NS5B RNA-dependent RNA polymerase. Despite inducible production of HCV core protein, in levels comparable to the full length construct, no detectable (−) strand synthesis was seen by either RT-PCR or RNase protection assay.

In the case of the deletion mutant, the inability to carry out successful (−) strand synthesis in the face of preserved structural protein synthesis and in the presence of an intact RNA template terminus strongly suggested that synthesis of antigenomic RNA requires the presence of HCV nonstructural proteins. The finding that equivalent levels of core protein were produced by the wild-type and mutant constructs suggested that the observed protein synthesis was most likely attributable to the initial effects of polyprotein translation by host ribosomes rather than the result of repeated rounds of viral genome replication.

Interferon-α directly and selectively inhibits HCV RNA and protein synthesis. Having established a successful binary cell-based expression system for (−) strand HCV RNA synthesis, the effects of potentially clinically active antiviral compounds on (−) strand synthesis was next examined. Interferon-α-2b (IFN-α), with or without ribavirin, is the only compound presently approved for the treatment of chronic HCV infection. Without an in vitro replication system for HCV, it is difficult to separate IFN-α's direct antiviral effects from its indirect effects, such as immunomodulatory effects. Therefore, the HCV replication system described herein was used to examine the direct effects of IFN-α on HCV replication.

Recombinant interferon-α-2b was added to the cell culture system used to generate replicative HCV, as described above, at doses predicted to have an antiviral effect in tissue culture. A dose-dependent inhibition of HCV (−) RNA synthesis by IFN-α was found at doses from 2000 to 15,000 units/well. These doses had no effect on RNA levels of actin and β-galactosidase in the RNase protection assay. When IFN-α's effects on protein synthesis were examined, a dose-responsive inhibition of HCV core protein synthesis by IFN-α was observed. This effect was substantially greater than IFN-α's modest effects on β-galactosidase levels. Actin protein synthesis was unaffected. Taken together, these data demonstrate that IFN-α exerts a direct antiviral effect on HCV RNA and protein synthesis.

The nucleoside analogue ribavirin is an approved agent, when given in conjunction with INF-α, for the treatment of chronic hepatitis C infection. While ribavirin did not appear to be effective against HCV in monotherapy, the drug's antiviral effects could be augmented by IFN (Davis et al., N. Engl. J. Med. 339:1493-1499, 1998; and Hoofnagle et al., J. Hepatol. 31:264-268, 1999). To determine whether this compound had direct anti-HCV activity, ribavirin was tested at doses of 10-200 mg/well. These doses are comparable to, and in excess of, those that produce clinically applicable concentrations. No inhibitory effect of ribavirin on HCV (−) RNA synthesis was found.

In some reports, amantadine demonstrated an antiviral effect against several RNA viruses, including HCV. When amantadine was tested at doses (1-100 mg/well) in excess of clinically relevant concentrations, it showed no activity against HCV (−) strand synthesis.

Neither ribavirin nor amantadine demonstrated direct activity against HCV (−) RNA synthesis, suggesting that any observed effect on the control of in vivo infection may be attributable to other actions, e.g., directly by increasing the HCV mutation rate or indirectly by an immunomodulatory mode of action. Further, when combined with the positive results obtained for IFN-α above, the studies described herein demonstrate that the HCV replication model can be used to determine whether candidate antiviral compounds have a direct affect on viral replication.

### Example 9

Candidate Antiviral Mutagens

To study the effect of known HCV antiviral agents on the HCV mutation rate, ribavirin or interferon-α-2b was added at the indicated doses to selected transfected/infected CV-1 cells 20 hours prior the introduction of vvT7. Twenty-four hours post-vvT7, cells were lysed and RNA was extracted by TRIzol (GIBCO/BRL), DNase I-treated (Roche Molecular Biochemicals), and phenol-chloroform extracted.

### Example 10

Reverse Transcription-PCR for Antiviral Mutagenesis Studies

RT-PCR was performed for the 5′UTR, core, E2-HVR1, NS5A and NS5B regions using region-specific primers:

5′ UTR: sense, nt 41-60: 5′CCCCTGTGAGGAACTACTGT-3′ (SEQ ID NO:3); antisense, nt 360-341: 5′-GGTGCACGGTCTACGAGACC-3′ (SEQ ID NO:4).

core: sense, nt 266-297: 5′-GGGTCGCGAAAGGCCTTGTGGTACTGCCTGAT-3′ (SEQ ID NO:5);, antisense nt 838-809: 5′-GTTGCATAGTTCACGCCGTCTTCCAGAACC-3′ (SEQ ID NO:6).

E2: sense nt 802-841: 5′-GCGTCCGGGTTCTGGAAGACGGCGTGAACTATGCAACA GG-3′ (SEQ ID NO:7); antisense, nt 1639-1600:5′AGGCTTTCATTGCAGTTCAAGGCCGT GCTATTGATGTGCC-3′ (SEQ ID NO:8).

NS5A: sense, nt 6853-6872: 5′-TGACGTCCATGCTCACTGAT-3′ (SEQ ID NO:9); antisense, nt 7176-7157: 5′-GAGACTTCCGCAGGATTTCT-3′ (SEQ ID NO:10).

NS5B: sense, nt 8245-8275: 5′-TGGGGATCCCGTATGATACCCGCTGCTTTGA-3′ (SEQ ID NO:11); antisense, nt 8645-8616: 5′-GGCGGAATTCCTGGTCATAGCCTCCGTGAA-3′ (SEQ ID NO:12).

RT was carried out on extracted RNA using AMV reverse transcriptase (Perkin-Elmer) using standard conditions. 25 cycles of PCR were carried out using 25 pmol each of the relevant sense and antisense primers, 0.5 mM of each dNTP, 1.5 mM MgCl2, and 0.5 units of Taq polymerase (Perkin-Elmer).

### Example 11

HCV RNA Quasispecies Analysis

Amplicons were cloned using the TOPO-PCR system (Invitrogen). Between 11 and 20 individual transformants corresponding to each region were sequenced bidirectionally by the ABI Prism automated sequencer, using primers flanking the insert. Synonymous and non-synonymous substitutions at variance from the parent pT7flHCV-Rz and pT7HCVDBglII-Rz sequences were determined and error generation rates calculated as nucleotide substitutions/total nucleotides sequenced. Sequences were aligned, and quasispecies diversity rates were compared between groups by using the statistical package SPSS 9.0.

### Example 12

HCV Quasispecies Generation is Template-Dependent

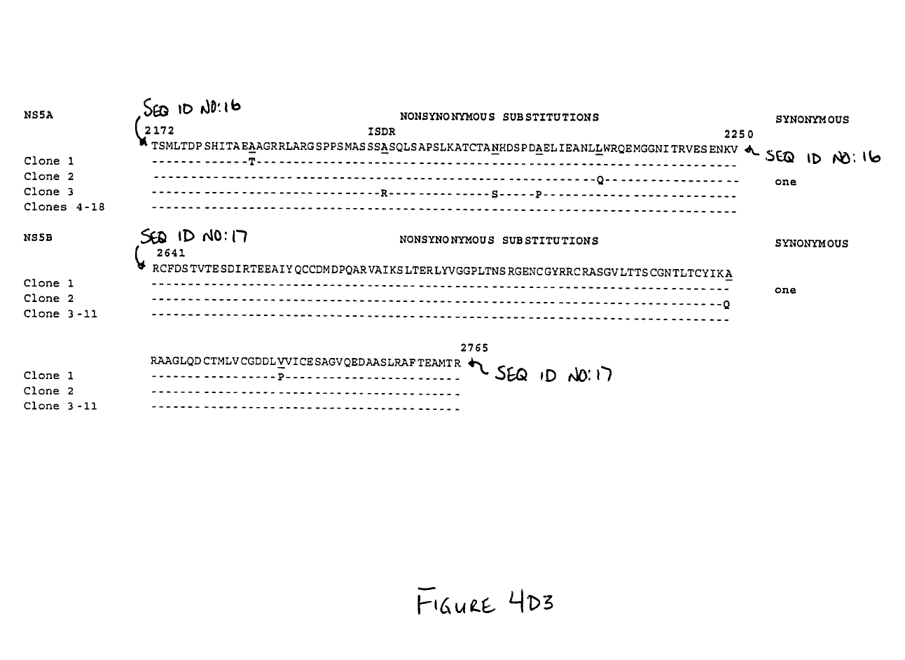

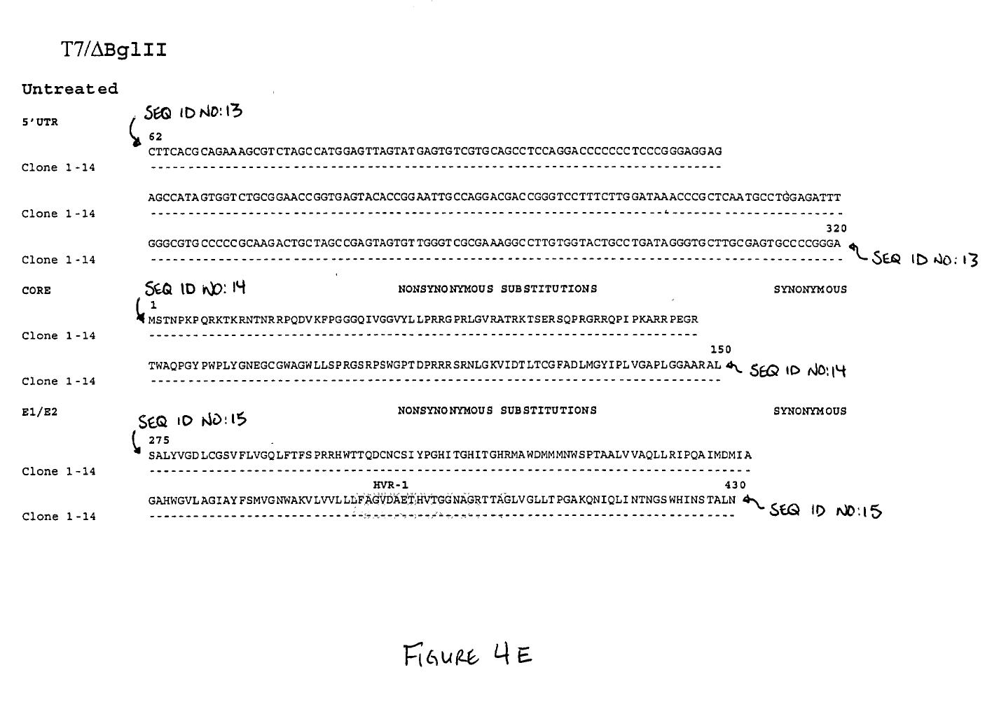

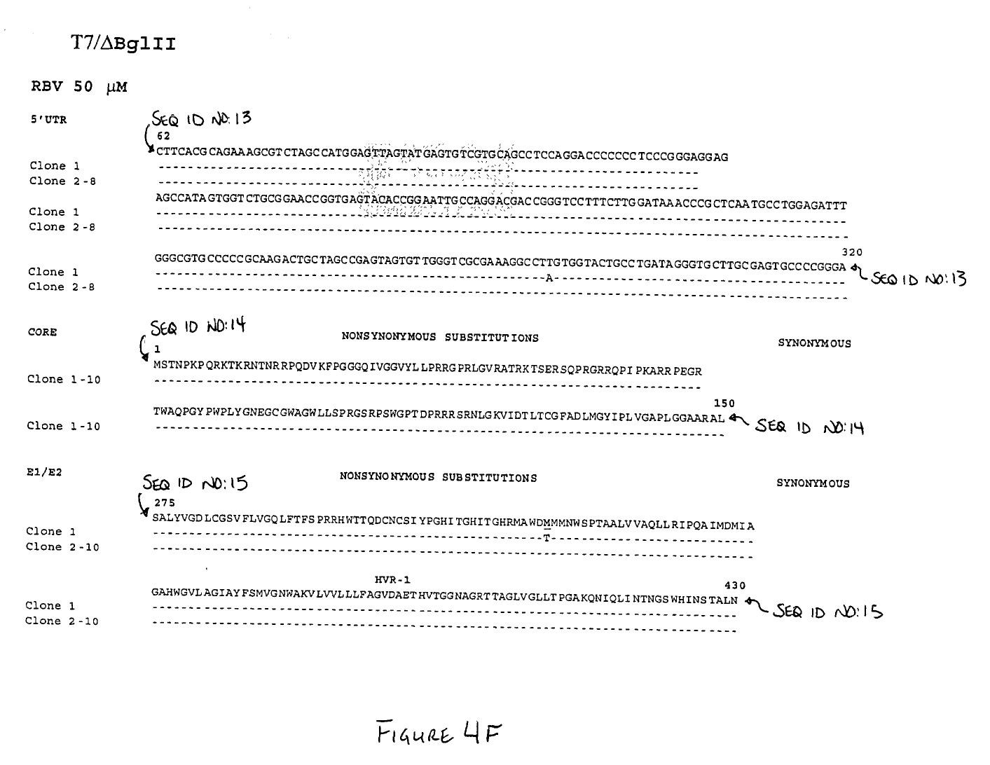

We used our full-length binary HCV expression system to demonstrate the successful synthesis of HCV (+) and (−) strand RNA and protein (see Chung et al. (2001), Proc. Natl. Acad. Sci. USA 98:9847-9852). In addition, we observed the generation of quasispecies corresponding to the E2-HVR1 region that was specific to cells treated with the full-length wildtype T7/pT7flHCV-Rz but not in cells treated with the mutant T7/pT7HCVDBglII-Rz lacking critical HCV RNA-dependent RNA polymerase sequences (see FIGS. 4A and E). These data strongly supported evidence of the action of the low-fidelity NS5B polymerase on the viral RNA template. We therefore asked whether the generation of quasispecies extended to other portions of the HCV genomic template.

In addition to E2 HVR-1, we examined sequences from the 5′ untranslated region, core, NS5A, and NS5B regions. Table 1 demonstrates the region-specific frequency of nucleotide substitutions corresponding to these regions. While we observed 9 nucleotide substitutions (4 nonsynonymous) in 4 independent clones for E2 HVR-1, and 5 substitutions (4 nonsynonymous) in NS5B, we found no substitutions in 5′UTR, core, and NS5A. In contrast, in the control samples, we observed no substitutions corresponding to the T7/pT7HCVDBglII-Rz infected-transfected cells in the 5′UTR, core and E2 HVR-1 regions (see FIG. 4E) and 2 substitutions in NS5B, one in core, and one in E2 HVR-1 corresponding to amplicons generated directly from the DNA template pT7flHCV-Rz. The DNA template was used as a control for Taq™ and sequencing errors for the nonstructural regions (NS5A and NS5B) not amplifiable from the deletion mutant pT7HCVDBglII-Rz. Thus, quasispecies generation is dependent on the template region selected. Further, quasispecies generation is not confined to E2 HVR-1, although this region displayed the greatest variability. Specifically, the error generation rate (number of substitutions/total number of nucleotides sequenced) was greatest for E2 HVR1 (1.7×10⁻³; p<0.04 by comparison to T7/pT7HCVDBglII-Rz, Fisher's exact test), followed by NS5B (0.7×10⁻³; p=NS).

TABLE 1

| | 5′ | | | | |
|---|---|---|---|---|---|
| | UTR | Core | E2 | NS5A | NS5B |
| T7/H77 | | | | | |
| CLONES (No) | 11 | 20 | 11 | 14 | 17 |
| MUTANT CLONES (%) | 0 | 0 | 4(36%) | 0 | 4(17%) |
| NONSYN/SYNON SUBST | 0 | 0 | 4/5 | 0 | 4/1 |
| ERROR GEN RATE (X 10⁻³) | 0 | 0 | 1.7* | 0 | 0.7 |
| H77 | | | | | |
| CLONES (No) | 11 | 11 | 11 | 12 | 12 |
| MUTANT CLONES (%) | 0 | 1 | 1 | 0 | 2 |
| NONSYN/SYNON SUBS | 0 | 1/0 | 1/0 | 0 | 2/0 |
| ERROR GEN RATE (X 10⁻³) | 0 | 0.1 | 0.1 | 0 | 0.4 |
| T7/Bgl II | | | | | |
| CLONES (No) | 14 | 11 | 11 | | |
| MUTANT CLONES (%) | 0 | 0 | 0 | | |
| NONSYN/SYNON SUBS | 0 | 0 | 0 | | |
| ERROR GEN RATE (X 10⁻³) | 0 | 0 | 0 | | |

*p < 0.04, Fisher's Exact Test

### Example 13

Ribavirin Increases HCV RNA Mutation Rate

We examined the effects of ribavirin (RBV) in excess of clinically relevant doses to determine whether this agent acts as an RNA mutagen. We used two doses that displayed the ability to inhibit HSV-1 plaque formation in CV-1 cells but had no effect on HCV replicative strand synthesis (see above). We found that RBV induces mutations across all regions of the HCV genome tested (see FIG. 4B). At the lower dose (50 mM), we found 15 nucleotide substitutions, 1 insertion, and 1 deletion in the HVR-1 region in 12 of 20 clones examined (1.7×10⁻³). Of greater interest, we found significant or near-significant increases in error generation in the 5′ UTR, core, NS5A, and NS5B regions (Table 2) compared to untreated HCV-expressing cells. For instance, in the core region, we found 12 substitutions and 4 insertions in 7 of 14 clones sequenced, where no errors were seen in the untreated cells (p<0.0006). The most significant differences were seen in regions that had previously demonstrated no sequence variation (core, 5′UTR, NS5A). These mutations were even more prominent when compared with a composite of the negative control T7/pT7-HCVDBglII (to encompass the 5′UTR and structural regions of the template) and direct PCR amplicons from the control plasmid pT7flHCV-Rz itself (to encompass the nonstrictural regions of the template). These differences are represented in Table 3.

TABLE 2
Comparison between RBV, IFN with T7/H77

| | 5′UTR | CORE | E2 | NS5A | NS5B |
|---|---|---|---|---|---|
| T7/H77 | | | | | |
| CLONES(#) | 11 | 20 | 11 | 14 | 17 |
| MUTANT CLONES (%) | 0 | 0 | 4(36%) | 0 | 4(17%) |
| NONSYN/SYN SUBS | 0 | 0 | 4/5 | 0 | 4/1 |
| ERROR GENRATE(X 10⁻³) | 0 | 0 | 1.7¹ | 0 | 0.7 |
| +REV 50 | | | | | |
| CLONES (#) | 15 | 14 | 20 | 20 | 18 |
| MUTANT CLONES (%) | 3(20%) | 7(50%) | 12(60%) | 5(20%) | 7/39%) |
| NONSYN/SYN SUBS | 0/3 | 8/4 | 11/4 | 12/0 | 9/4 |
| ERROR GEN RATE(X 10⁻³) | 0.7 | 1.9² | 1.5 | 2.5³ | 1.7 |
| | | 4 nt-insertions | 1 nt-insertion | | |
| | | | 1 nt-deletion | | |
| +RBV 400 | | | | | |
| CLONES (#) | 14 | 20 | 20 | 14 | 17 |
| MUTANT CLONES (%) | 3(21%) | 4(20%) | 7(35%) | 2(14%) | 6(35%) |
| NONSYN/SYN SUBS | 0/3 | 5/1 | 9/4 | 3/0 | 5/2 |
| ERROR GEN RATE(X 10⁻³) | 0.7 | 0.6⁴ | 1.2 | 0.9 | 1 |
| +IFN 100,000U | | | | | |
| CLONES(#) | 13 | 11 | 12 | 18 | 11 |
| MUTANT CLONES (%) | 0 | 4(36%) | 10(83%) | 3(16%) | 2(18%) |
| NONSYN/SYN SUBS | 0 | 2/2 | 10/6 | 5/1 | 3/1 |
| ERROR GEN RATE(X 10⁻³) | 0 | 0.8⁵ | 2.8⁶ | 1.4 | 0.9 |
| | | | 4 nt-insertions | | |

¹p < 0.04;
²p < 0.00006;
³p < 0.06;
⁴p < 0.05;
⁵p < 0.01;
⁶p < 0.03

TABLE 3
Comparison between RBV, IFN and controls

| | 5′UTR | CORE | E2 | NS5A | NS5B |
|---|---|---|---|---|---|
| 7/BgI 11^{A} | | | | | |
| CLONES(#) | 14 | 11 | 11 | 12 | 17 |
| MUTANT CLONES (%) | 0 | 0 | 0 | 0 | 2 |
| NONSYN/SYN SUBS | 0 | 0 | 0 | 0 | 2/0 |
| ERROR GEN RATE(X 10⁻³) | 0 | 0 | 0 | 0 | 0.4 |
| RBV 50 | | | | | |
| CLONES (#) | 15 | 14 | 20 | 20 | 18 |
| MUTANT CLONES (%) | 3(20%) | 7(50%) | 12(60%) | 5(20%) | 7(39%) |
| NONSYN/SYN SUBS | 0/3 | 8/4 | 11/4 | 12/0 | 9/3 |
| ERROR GEN RATE(X 10⁻³) | 0.7 | 1.9¹ | 1.5² | 2.5³ | 1.7 |
| | | 4 nt-insertions | 1 nt-insertion | | |
| | | | 1 nt-deletion | | |
| +RBV 400 | | | | | |
| CLONES (#) | 14 | 20 | 20 | 14 | 17 |
| MUTANT CLONES (%) | 3(21%) | 4(20%) | 7(35%) | 2(14%) | 6(35%) |
| NONSYN/SYN SUBS | 0/3 | 5/1 | 7/5 | 3/0 | 5/2 |
| ERROR GEN RATE(X 10⁻³) | 0.7 | 0.6 | 1.2⁴ | 0.9 | 1 |
| +IFN 100,000U | | | | | |
| CLONES(#) | 13 | 11 | 12 | 18 | 11 |
| MUTANT CLONES (%) | 0 | 4(36%) | 10(83%) | 3(16%) | 2(18%) |
| NONSYN/SYN SUBS | 0 | 2/2 | 10/6 | 5/1 | 3/1 |
| ERROR GEN RATE (X 10⁻³) | 0 | 0.8 | 2.8⁶ | 1.4 | 0.9 |
| | | | 4 nt-insertions | | |

Regions 5′UTR, Core, E1/E2 are compared with T7/Bgl II. Regions NS5A and NS5B are compared with H77.
¹p < 0.008;
²p < 0.001;
³p < 0.05;
⁴p < 0.06;
⁵p < 0.04;
⁶p < 0.00007

At higher doses of ribavirin (400 mM), we continued to observe error generation throughout the viral genome, but to a lesser degree than that observed at the lower dose (see FIG. 4C), although the differences were not statistically significant (Table 2). The degree of variability was evenly balanced across all regions (error generation 0.6-1.2×10⁻³). Again, the most significant changes at this RBV dose from untreated replicating HCV RNA were observed in the core region (p<0.05). Table 3 also demonstrates these differences when compared to the composite control sequences.

### Example 14

Interferon-α-2b increases the HCV RNA mutation rate

We examined the effect of interferon-α-2b (IFN) on HCV RNA sequences generated by our system. When a dose of IFN (100,000 U) sufficient to decrease HCV RNA synthesis (see above) was administered to cells in the presence of vvT7 and pT7-flHCV-Rz, we observed an increase in error generation in 4 of the 5 regions sequenced (Table 2; see also FIG. 4D). HCV core, E2 HVR-1, NS5A and NS5B all showed increased rates of mutation. In the case of core and E2 HVR-1, this difference was statistically significant over untreated cells expressing wildtype full length RNA. Only 5′ UTR sequences remained invariant in the presence of IFN. This error generation was even more apparent when compared to the composite control sequences (Table 3). Overall, the mutation rates attributable to ribavirin and interferon treatment were significantly different when compared to untreated T7/pT7-flHCVRz transfected-infected CV-1 cells (c2 with Yates correction). Similarly, the portion of mutant clones from each treated group was similar (about 30%), but the number of variant nucleotides per clone differed. When the error generation rate was averaged across all regions examined, we found an overall mutation rate of 0.9-3.2×10⁻⁴/site.

### Example 15

Synonymous to Nonsynonymous Ratio and G-to-A or C-to-T Transition Mutations

Under untreated conditions (T7/pT7flHCV-Rz), we observed a dN/dS (nonsynonymous/synonymous mutation) of 1.33 (8:6) for all regions tested (Table 4). This ratio increased to 2.85 with ribavirin at 50 mM, 1.82 with ribavirin at 400 mM, and 2.0 with IFN-α. We observed a large number of transition mutations in the presence of ribavirin at both doses (Tables 6 and 7), including G to A and C to U transitions that would be predicted by in vitro ribavirin incorporation experiments (Crotty et al. (2000), Nat. Med. 6:1375-1379), although C to U transitions were observed in the E2 HVR-1 in the absence of treatment. We also observed a number of transition mutations with interferon treatment, although there were fewer absolute and relative G to A and C to U transitions observed under these conditions (Table 8). These differences were not statistically significant. Characterization of the direct effects of the observed substitutions, insertions and deletions revealed that at least 5 mutations (2 insertions, 1 deletion, 2 substitutions) led to premature stop codons within the directly sequenced regions (Tables 4, 6, and 8; see also FIG. 4), and so would be predicted to result in nonviable proteins.

TABLE 4

| | Total | Mutant | Total | | |
|---|---|---|---|---|---|
| | Clones | Clones | Mutations | Non-Syn/Syn | |
| | (#) | (%) | (all regions) | Substitutions | value* |
| T7/H77 | 73 | 8(11%) | 14 | 8/6 | |
| RBV 50 | 87 | 29(33%) | 54 | 40/14 | <0.001 |
| RBV 400 | 85 | 25(30%) | 31 | 20/11 | <0.008 |
| IFN | 65 | 19(30%) | 30 | 20/10 | <0.01 |
| 100,000 | | | | | |

TABLE 5
Transition Mutations T7/H77

| | G to A | A to G | T to C | C to T | Comments |
|---|---|---|---|---|---|
| 5′UTR | | | | | |
| CORE | | | | | |
| E2 | | 1 | 1 | 4 | |
| NS5A | | | | | |
| NS5B | | 2 | | 2 | |

TABLE 6
Transition Mutations RBV 50

| | G to A | A to G | T to C | C to T | Comments |
|---|---|---|---|---|---|
| 5′UTR | | | 2 | | |
| CORE | | | 2 | 2 | 2 insertions |
| | | | | | (resulting in stop codons) |
| E2 | 1 | 4 | 6 | 1 | 2 insertions |
| | | | | | (resulting in stop codons) |
| NS5A | | 1 | | 1 | |
| NS5B | 4 | 2 | | 1 | |

TABLE 7
Transition Mutations RBV 400

| | G to A | A to G | T to C | C to T | Comments |
|---|---|---|---|---|---|
| 5′UTR | 1 | 1 | | | |
| CORE | 1 | 1 | 1 | 2 | |
| E2 | 1 | 3 | 1 | 2 | 2 insertions |
| NS5A | | | | | |
| NS5B | 1 | 2 | 2 | | |

TABLE 8
Transition Mutations IFN 100,000U

| | C to A | A to G | T to C | C to T | Comments |
|---|---|---|---|---|---|
| 5′UTR | | | | | |
| CORE | | | 2 | | |
| E2 | | 4 | 9 | 2 | 4 insertions; 1 stop codon |
| NS5A | 2 | 1 | 1 | | |
| NS5B | | | | | |

### Example 16

Alteration of the HCV Mutation Rate

Using a novel HCV RNA replication system capable of recapitulating the early steps in the HCV lifecycle, it was found that RNA quasispecies generation is observed in other regions of the HCV genome, but mutation generation rates are highly variable. Specifically, significant error generation above that observed with the control plasmid lacking critical nonstructural region sequences necessary for RNA replication was most frequently observed in the E2 HVR-1 region, but was also seen to a lesser extent in the NS5B region. However, mutations were not observed in 3 other regions: 5′UTR, core, and NS5A. These findings, produced in the complete absence of immune selection pressure, have a number of implications for the HCV NS5B RNA-dependent RNA polymerase.

First, the finding of variable error generation suggests that the low fidelity attributable to the NS5B enzyme is template-dependent. That the highest error frequency occurred in a region (HVR-1) exhibiting the greatest quasispecies diversity from in vivo isolates suggests that in addition to immune selection pressure, there is intrinsic quasispecies generation as a result of impaired template copying. This miscopying is likely to be a result of complex RNA template secondary structure, such as that observed in the E2 HVR-1 region. Hence, two independent mechanisms appear to underlie the observed hypervariablity of this region. We also found variability in the NS5B region; in this regard, it is notable that RNA secondary structure analyses have demonstrated significant secondary structure in this region as well (see Walewski et al. (2001), RNA 7:710-721).

Second, the finding of quasispecies in a short-term expression system suggests that the regional accumulation of mutations is likely to be very high. Hence, it is very likely that the observed substitutions, insertions, and deletions will frequently lead to nonviable polyprotein. Thus, the mutation rates here can offer estimates of the NS5B error generation rate, but not of the true viable quasispecies generation rate.

Using our replication system, we found that RBV increases error generation, especially in otherwise invariant regions, suggesting that it acts as an RNA mutagen in vivo.

In this regard, we confirm the findings of Crotty et al. (supra). Our findings differ in several respects, however. First, the observed RNA mutation rate did not approach those described by Crotty et al. (supra). Second, we found no direct decrease in HCV (−) RNA attributable to the use of RBV at either of the doses tested, despite the observation of its activity against HSV-1 in the same cell line. Third, there was not a demonstrable dose-responsiveness of mutations observed. We speculate that RBV increases the HCV RNA mutagenesis rate, but not to the brink of the catastrophic error rates described with poliovirus and thus without demonstrable inhibitory effects on viral RNA synthesis in our assay. In this regard, ribavirin may function against HCV in a manner more analogous to its action against the related vinis GBV-B. Virions from cultured primary hepatocytes treated with RBV have reduced infectivity, but RBV appears not to have inhibitory effects on GBV in vivo (Lanford et al., J. Virol. 75:8074-8081).

We speculate that the failure to observe a linear RBV dose response effect may reflect threshold effects obtained at lower doses.

We observed substitutions in areas previously invariant at both doses of RBV used. In particular, the finding of RBV-induced NS5A substitutions in the interferon sensitivity determining region defined by Enomoto et al. (1996), N. Engl. J. Med. 334:77-81, raises the possibility that RBV could enhance generation of interferon-sensitive quasispecies. While additional RNA inhibitory effects were not seen when RBV was added to IFN in our system, this inhibition may be revealed only with more long-term expression. The generation of IFN-sensitive quasispecies might provide an additional mechanism to explain the significant enhancement of IFN's antiviral effect by the addition of RBV in vivo.

We also observed an increase in quasispecies generation across all coding regions by treatment of our system with IFN. We found this of interest, since we and others have previously demonstrated IFN's direct inhibitory effect on HCV RNA and protein synthesis (Chung et al., supra, and Blight et al. (2000), Science 290:1972-1975). These data suggest that in addition to its well-described effects on RNA synthesis, IFN may also increase generation of nonviable RNA species. We speculate that this occurs not through RNA mutagenesis, per se, but rather indirectly via alteration of availability of template and host factors critical to successful HCV RNA polymerization.

It has been hypothesized that nonsynonymous mutations are a reflection of exogenously applied selection pressure rather than the result of enzymatic activity (Ray et al. (2000), J. Virol. 74:3058-3066). However, in our system we consistently observed a nonsynonymous/synonymous mutation ratio in excess of one. This finding would not be predicted by chance, given the likelihood that a random mutation would be predicted to produce synonymous substitutions. One possible explanation might lie in constraints imposed by the existence of cryptic alternate open reading frames, such as that described within the core region (Walewski et al., supra). It is important to note that not all of the RNA species generated by our system are destined to become viable quasispecies. Indeed, those producing true nonsynonymous substitutions have a significantly higher likelihood of being nonviable and thus never incorporated into mature virion. The RNA species identified in our system do not distinguish between viable and nonviable sequences, and thus do not necessarily reflect the balance of circulating quasispecies in vivo.

Because our system relies exclusively on cultured epithelial cell lines and thus does not introduce immune selection pressure, our findings are likely to reflect the in vivo fidelity and species generation of the wildtype NS5B RNA-dependent RNA polymerase. They suggest not only that the RNA template may be susceptible to miscopying, but also that this susceptibility is enhanced in regions likely to lead to amino acid substitutions. These findings could reflect an evolutionary template strategy for further generation of sequence diversity, even at the cost of increased generation of nonviable species.

A number of studies have examined the longitudinal mutation rate of HCV sequences from infected humans and chimpanzees. Estimates have ranged from 0.4 to 1.9×10−3 bases per site per year. The findings in our study suggest that the mutation rate attributable to NS5B miscopying is substantially higher, leading to generation of quasispecies in some regions over a short term. We hypothesize that the nonviability rate is significant and accounts for the more modest numbers observed over time in infected hosts.

Our system will serve as a potentially useful tool for assessments of cell-based activity of the NS5B polymerase, not only with regard to error generation, but also other enzymatic properties, including processivity and kinetics. It may also serve as a potentially rich source of in vivo replication complexes, with characterization of other members of this multiprotein assembly possible. Finally, the ability of our system to detect RNA mutations generated by potentially mutagenic compounds suggests that it will be a useful assay for screening of potential antiviral compounds with activity against HCV. This will be especially true of compounds with affinity for the NS5B polymerase, including nucleoside analogues.

What is claimed is:

1. A method of identifying a compound that increases the mutation rate of hepatitis C virus, comprising:
providing a test cell containing a nucleic acid molecule comprising a first nucleotide sequence consisting of an infectious hepatitis C viral genome, or a DNA copy thereof; a second nucleotide sequence consisting of a ribozyme, or a DNA copy thereof; and an inducible promoter operably linked to the first and second nucleotide sequences, the ribozyme being configured to remove a 3′ sequence unnecessary for replication of the infectious hepatitis C viral genome from a transcript initiated by the inducible promoter;
inducing the inducible promoter;
contacting the test cell with a candidate compound; and
detecting an increase in hepatitis C virus quasispecies produced by the cell in the presence of the candidate compound, as compared to hepatitis C virus quasispecies produced in the absence of the candidate compound, wherein said increase in hepatitis C virus quasispecies indicates that the candidate compound increases the mutation rate of hepatitis C virus.

2. The method of claim 1, wherein the candidate compound is not a ribonucleoside analog.

3. The method of claim 1, wherein the inducible promoter is a T7 promoter.

4. The method of claim 3, wherein the inducing step is performed by expressing a T7 RNA polymerase in the cell.

5. The method of claim 1, wherein detecting an increase in hepatitis C virus quasispecies comprises amplifying at least one region of a hepatitis C viral genome by RT-PCR to form a RT-PCR product.

6. The method of claim 5, wherein the region comprises the 5′UTR, Core, E1, E2, NS5A, or NS5B region of the hepatitis C viral genome.

7. The method of claim 6, wherein the region comprises the Core region of the hepatitis C viral genome.

8. The method of claim 6, wherein the region comprises the E1 or E2 region of the hepatitis C viral genome.

9. The method of claim 6, wherein the region comprises the NS5A region of the hepatitis C viral genome.

10. The method of claim 6, wherein the region comprises the NS5B region of the hepatitis C viral genome.

11. The method of claim 5, wherein the RT-PCR product is analyzed by nucleic acid sequencing.

12. The method of claim 11, wherein nucleic acid molecules present in the RT-PCR product are sequenced individually.

13. The method of claim 12, wherein at least ten nucleic acid molecules are sequenced.

14. The method of claim 5, wherein the RT-PCR product is analyzed by restriction fragment length polymorphism analysis.

15. The method of claim 14, wherein nucleic acid molecules present in the RT-PCR product are analyzed individually.

16. A method of identifying a compound that increases the mutation rate of hepatitis C virus, comprising:
providing a test cell containing a nucleic acid molecule comprising a first nucleotide sequence consisting of an infectious hepatitis C viral genome, or a DNA copy thereof; a second nucleotide sequence consisting of a ribozyme, or a DNA copy thereof; and an inducible promoter operably linked to the first and second nucleotide sequences, the ribozyme being configured to remove a 3′ sequence unnecessary for replication of the infectious hepatitis C viral genome from a transcript initiated by the inducible promoter;
inducing the inducible promoter;
contacting the test cell with a candidate compound that is not a ribonucleoside analog; and
detecting an increase in hepatitis C virus quasispecies produced by the cell in the presence of the candidate compound, as compared to hepatitis C virus quasispecies produced in the absence of the candidate compound, wherein an increase in hepatitis C virus quasispecies indicates that the candidate compound increases the mutation rate of hepatitis C virus.

17. The method of claim 16, wherein the inducible promoter is a T7 promoter.

18. The method of claim 17, wherein the inducing step is performed by expressing a T7 RNA polymerase in the cell.

19. The method of claim 16, wherein detecting an increase in hepatitis C virus quasispecies comprises amplifying at least one region of a hepatitis C viral genome by RT-PCR to form a RT-PCR product.

20. The method of claim 19, wherein the region comprises the 5′UTR, Core, E1, E2, NS5A, or NS5B region of the hepatitis C viral genome.

21. The method of claim 20, wherein the region comprises the Core region of the hepatitis C viral genome.

22. The method of claim 20, wherein the region comprises the E1 or E2 region of the hepatitis C viral genome.

23. The method of claim 20, wherein the region comprises the NS5A region of the hepatitis C viral genome.

24. The method of claim 20, wherein the region comprises the NS5B region of the hepatitis C viral genome.

25. The method of claim 19, wherein the RT-PCR product is analyzed by nucleic acid sequencing.

26. The method of claim 25, wherein nucleic acid molecules present in the RT-PCR product are sequenced individually.

27. The method of claim 26, wherein at least ten nucleic acid molecules are sequenced.

28. The method of claim 19, wherein the RT-PCR product is analyzed by restriction fragment length polymorphism analysis.

29. The method of claim 28, wherein nucleic acid molecules present in the RT-PCR product are analyzed individually.
